(19) United States
(12) Patent Application Publication
(10) Pub. No.: US 2026/0066119 A1
(43) Pub. Date: Mar. 5, 2026

(54) SYSTEM AND METHOD FOR ALGORITHMIC DIAGNOSTICS FOR EFFICIENT PRESCRIPTION OF TREATMENTS FOR B-SNIP PSYCHOSIS BIOTYPES

(71) Applicants: University of Georgia Research Foundation, Inc., Athens, GA (US); Yale University, New Haven, CT (US); The University of Chicago, Chicago, IL (US); The Board of Regents of The University of Texas System, Austin, TX (US); Beth Israel Deaconess Medical Center, Boston, MA (US)

(72) Inventors: Brett A. Clementz, Athens, GA (US); Robert David Gibbons, Chicago, IL (US); Ishanu Chattopadhyay, Georgetown, KY (US); Jennifer E. McDowell, Athens, GA (US); David Alan Parker, Athens, GA (US); Elliot S. Gershon, Chicago, IL (US); Godfrey D. Pearlson, Bloomfield, CT (US); Elena Ivanovna Ivleva, Dallas, TX (US); Sarah Keedy, Chicago, IL (US); Matcheri S. Keshavan, Westwood, MA (US); Carol A. Tamminga, Dallas, TX (US)

(21) Appl. No.: 19/314,645

(22) Filed: Aug. 29, 2025

Related U.S. Application Data
(60) Provisional application No. 63/688,599, filed on Aug. 29, 2024.

(51) Int. Cl. G16H 50/20; G06N 5/01; G06N 20/20; G16H 10/60; G16H 20/00; G16H 50/30; G16H 50/70

(52) U.S. Cl. CPC G16H 50/20; G06N 5/01; G06N 20/20; G16H 10/60; G16H 20/00; G16H 50/30; G16H 50/70

(57) ABSTRACT

A method and system for Adaptive Diagnostics for the Efficient Prescription of Treatments (ADEPT), which efficiently diagnoses an idiopathic psychosis patient and improves treatment targeting for that patient. ADEPT can be divided into two versions that use different inputs: ADEPT for measuring clinical characteristics of a patient (ADEPT-CLIN) and ADEPT for measuring cognitive performance (ADEPT-COG). The ADEPT systems identify Biotypes by accessing the database to match the characteristics of the patient to similar patients in that database. For ADEPT-CLIN, the patient's Biotype is based on similarity to existing patients on clinical characteristics. For ADEPT-COG, the patient's Biotype is based on similarity to existing patients on clinical, behavioral, motor inhibition, and cognitive features. Biotypes are used to implement targeted treatment for an individual patient. ADEPT is continuously re-trained using new cases and novel laboratory tests to improve precision of Biotypes diagnosis and the accuracy of selecting treatments for individual patients.

### CROSS-REFERENCE TO RELATED APPLICATIONS

This application claims the benefit of and priority to U.S. Application No. 63/688,599, filed on Aug. 29, 2024, which is hereby incorporated herein by reference in its entirety.

### STATEMENT REGARDING FEDERALLY SPONSORED RESEARCH

This invention was made with government support under R01 MH124805, R01 MH124806, R01 MH124813, R01 MH127179, R01 MH127158, R01 MH124802, R01 MH077945, R01 MH096957, R01 MH078113, R01 MH096942, R01 MH096900, R01 MH094172, R21 MH126398, MH124804, MH127162, and MH103368 awarded by the National Institutes of Health. The government has certain rights in the invention.

### FIELD OF THE INVENTION

The invention is generally in the field of computer-implemented methods and systems for neurobiological diagnosis and/or treatment of serious psychiatric conditions, particularly neurobiological diagnosis and/or treatment of psychosis (e.g., idiopathic psychosis) using adaptive diagnostic algorithms preferably trained on clinical information, cognitive information, and/or behavioral information to recognize one or more subtypes of psychosis.

### BACKGROUND OF THE INVENTION

Many serious psychiatric conditions lack objectively measurable physical criteria for diagnosis. They are identified using only clinical features, which depend on reports by the patient and informants and the proper interpretation and belief in those reports by the evaluating clinical staff. The lack of objective extra-clinical and measurable criteria hinders diagnostic validity, understanding of disease mechanisms, and the appropriate treatment of many persons with serious psychiatric conditions.

Accordingly, a need arises for techniques that can accurately and objectively diagnose certain types of serious psychiatric conditions with specific neurobiological deviations and treatment targets, such as among persons with psychosis.

### SUMMARY OF THE INVENTION

The Bipolar-Schizophrenia Network for Intermediate Phenotypes (B-SNIP) searched for neurobiological similarity among persons with a psychosis of unknown origin, regardless of their clinical diagnosis, like schizophrenia, schizoaffective disorder, or bipolar disorder with psychosis. For initial screening, patients are identified as having an psychosis based on, for instance, interview or questionnaires. At this point, the specific psychosis diagnosis is unknown, much like the specific disease of a person with a fever is unknown. Patients are then administered laboratory tests spanning levels of analysis and brain functions that distinguish psychosis subgroups. Cognitive performance, saccadic eye movements, and EEG signals are measured on a patient. Principal component analysis is applied to the measured signals to determine the most significant components. Numerical taxonomy identifies clusters of patients who perform similarly across those measured signals.

The outcome is B-SNIP psychosis Biotypes that do not map to diagnoses such as schizophrenia, schizoaffective disorder, or bipolar disorder with psychosis. A patient can have, for instance, Biotype-1 (BT1) or Biotype-2 (BT2) or Biotype-3 (BT3). Biotype diagnoses of idiopathic psychosis patients have treatment targets that follow directly from those diagnoses. Those treatment targets are not derivable from any other currently available approach to idiopathic psychosis diagnosis.

It is impractical for many clinical and research sites to collect and quantify the full B-SNIP biomarker panel. The present innovative work efficiently assigns an individual to their B-SNIP psychosis Biotype without the need to collect an extensive battery of laboratory tests. In an aspect, this application is directed towards a decision tree algorithm that efficiently diagnoses neurobiologically defined B-SNIP psychosis Biotypes with greater than 95% accuracy. These Biotypes have specific treatment targets not derivable from any other available approach.

In one aspect, the platform, called Adaptive Diagnostics for the Efficient Prescription of Treatments (ADEPT), interacts with the processed B-SNIP biomarker database. In another aspect, there are two versions of ADEPT. One version requires only clinical interview and evaluation and is called ADEPT-CLIN. The other version requires the addition of behavioral and cognitive performance assessments and is called ADEPT-COG. The present disclosure describes both versions and their methods. The techniques described herein comprise efficient, adaptive methods for diagnosing a B-SNIP psychosis Biotype in a patient that can be used in any clinical or research setting. The ability to efficiently assign a patient to their Biotype with such high accuracy is a surprising outcome of this innovative work and speaks to the validity of the implemented clinical and laboratory evaluations.

### BRIEF DESCRIPTION OF THE DRAWINGS

The file of this application contains drawings/photographs executed in color. Copies of this patent application publication with color drawing(s)/photograph(s) will be provided by the Office upon request and payment of the necessary fee.

So that the way the above recited steps and implementations of the present disclosure can be understood in detail, a more particular description of the innovative work, briefly summarized above, may be had by reference to embodiments, some of which are illustrated in the appended drawings. It is to be noted, however, that the appended drawings illustrate only typical embodiments of this innovative work, and the innovative work may admit to other equally effective embodiments. For instance, the ADEPT algorithm is continually re-trained with new cases and new measures based on accumulated experience and analytical expertise of the inventors.

FIG. 1 illustrates the global biomarker measure groups that differentiate and define B-SNIP psychosis Biotypes and the relationship of those same measures to DSM diagnoses. The figure illustrates that (i) B-SNIP Biotypes have differentiating neurobiological features and treatment targets but (ii) DSM diagnoses, part of the current standard of care, do not provide the same neurobiological treatment targets.

FIG. 2 illustrates the number of Biotype cases with different DSM diagnoses and the number of DSM diagnoses with different Biotypes. This shows that Biotypes do not recapitulate DSM diagnoses and DSM diagnoses are spread across B-SNIP Biotypes.

FIG. 3 illustrates the consistency of cognition and behavioral outcomes across two separate samples. This consistency supports practical application and implementation of the ADEPT algorithm.

FIG. 4 illustrates the consistency of ERP measurements across two separate samples. This consistency supports practical application and implementation of the ADEPT algorithm.

FIG. 5 illustrates the consistency of the Biotypes outcome as a function of sample sizes, which shows the number of participants that are preferably utilized to get a stable biomarker diagnostic system. A consistent outcome is attainable only using the B-SNIP biomarker panel and large numbers of psychosis persons in the computations. This shows part of the requirements for the successful development of a clinically valid and implementable neurobiological diagnostic procedure for idiopathic psychosis. It also shows that the inventors have a sufficiently large sample for deriving a stable B-SNIP psychosis Biotypes solution, which supports the implementation of the ADEPT algorithm.

FIG. 6 illustrates DSM versus Biotype group differences in effect size units and the integrated separation of DSM versus Biotype groups using multiple measures. Biotype categories show larger effect sizes than DSM categories. This illustrates that only B-SNIP psychosis Biotypes yield distinctive pathophysiological signatures with identifiable treatment targets. This supports the importance of the ADEPT algorithm for efficiently diagnosing B-SNIP psychosis Biotypes.

FIG. 7 illustrates external validation (construct validity) of the electrophysiological characteristics of B-SNIP psychosis Biotypes. This supports the importance of the ADEPT algorithm for efficiently diagnosing B-SNIP psychosis Biotypes.

FIG. 8 illustrates the external validation (construct validity) structural anatomical (hippocampal complex) characteristics of B-SNIP psychosis Biotypes. This supports the importance of the ADEPT algorithm for efficiently diagnosing B-SNIP psychosis Biotypes.

FIG. 9 shows raincloud plots from the analysis of GMD classifiers for the (a) Biotype group 1 (B1) vs control (CON), (b) biotype group 2 (B2) versus control (CON), and (c) Biotype group 3 (B3) versus control (CON). In each panel, the dots represent balanced classifier accuracy for each of 1000 iterations, the density plot shows the distribution of accuracy values across iterations, and the black dot and line reflects the mean and 99.17% interval of the accuracy values, respectively. Overall accuracy reflects on the two groups in the model (e.g., overall accuracy for the B1 model is the combined accuracy of B1 and CON cases). The other columns of the figure reflect accuracy for individual groups. Note that accuracy for groups not included in the training model (e.g., B2 and B3 for the B1 model), the “accuracy” value reflects the rate of classifier guesses for being in the psychosis group (B1). It also illustrates the diagnostic value of an MRI-based machine learning classifier specifically for B-SNIP psychosis Biotype-1 (BT1). This MRI-based algorithm illustrates a unique complement to the diagnosis of B-SNIP psychosis Biotype-1 (BT1). This supports the importance of the ADEPT algorithm for efficiently diagnosing B-SNIP psychosis Biotypes.

FIG. 10 illustrates the external validation of sensory training treatment implications for B-SNIP psychosis Biotypes. Level of performance on a tone matching test (y-axis) is shown as a function of eight training days (x-axis) for three different task difficulty levels. BT1 are the only group that improve over training days, consistent with B-SNIP's treatment expectation. BT2 got worse over time and BT3, with the best overall performance, did not improve with training. This illustrates that B-SNIP psychosis Biotypes provide an implementable sensory training treatment target for BT1 that is not derivable from any other existing approach to diagnosis of persons with an idiopathic psychosis. They also illustrate that not using B-SNIP psychosis Biotypes yields suboptimal care for individual idiopathic psychosis patients. This supports the importance of the ADEPT algorithm for efficiently diagnosing B-SNIP psychosis Biotypes.

FIG. 11 Illustrates the external validation (construct validity) of clozapine treatment implications for B-SNIP psychosis Biotypes. Level of performance in relation to healthy persons is shown for psychosis Biotypes (y-axis) across biomarker targets (x-axis) as a function of on or off clozapine. The bar charts illustrate that B-SNIP psychosis Biotypes provide implementable clozapine treatment targets for BT1 (IEA) or BT3 (SNR or signal-to-noise and BACS or general cognitive performance) not derivable from any other existing approach to diagnosis of persons with an idiopathic psychosis. They also illustrate that not using B-SNIP psychosis Biotypes yields suboptimal care for individual patients. This supports the importance of the ADEPT algorithm for efficiently diagnosing B-SNIP psychosis Biotypes.

FIG. 12 illustrates Biotype group differences in effect size units across multiple laboratory tests. B-SNIP psychosis Biotypes yield distinctive pathophysiological signatures with clear treatment targets. This supports the importance of the ADEPT algorithm for efficiently diagnosing B-SNIP psychosis Biotypes.

FIG. 13 illustrates the ADEPT-CLIN and ADEPT-COG accuracy of Biotype or DSM classification in out-of-sample runs (y-axis) as a function of mean number of features used in the decision (x-axis). This shows the accuracy of the three group decisions (BT1 or BT2 or BT3 for Biotypes; schizophrenia or schizoaffective disorder or bipolar disorder with psychosis for DSM). The shaded area is the 99% confidence interval. The accuracy of ADEPT-CLIN is displayed based on 58 clinical features alone. The accuracy of ADEPT-COG is displayed based on 76 clinical and cognitive features. The red arrow highlights the gain in accuracy for Biotypes when using the additional ADEPT-COG features. The shaded area is the 99% confidence interval.

FIG. 14 illustrates the relative importance in ADEPT-CLIN of every one of the 58 items for Biotype prediction (carrying out one-vs-all classification). PANSS=Positive and Negative Symptom Scale; MADRS=Montgomery-Asberg Depression Rating Scale; Young=Young Mania Rating Scale; SFS=Birchwood Social Functioning Scale. BT1=Biotype-1; BT2=Biotype-2; BT3=Biotype-3.

FIG. 15 illustrates the relative importance in ADEPT-CLIN of every one of the 58 items for DSM prediction (carrying out one-vs-all classification). PANSS=Positive and Negative Symptom Scale; MADRS=Montgomery-Asberg Depression Rating Scale; Young=Young Mania Rating Scale; SFS=Birchwood Social Functioning Scale. SZ=schizophrenia; SAD=schizoaffective disorder; BDP=bipolar disorder with psychosis.

FIG. 16 illustrates the ADEPT-CLIN sensitivity, specificity, and AUC (area under the curve) of out of-sample tests for differentiating BT1 (left figure), BT2 (middle figure), or BT3 (right figure) from the other groups using an efficient set of clinical features.

FIG. 17 illustrates the ADEPT-CLIN sensitivity, specificity, and AUC (area under the curve) of out of-sample tests for differentiating schizophrenia (left figure), schizoaffective disorder (middle figure), or bipolar disorder with psychosis (right figure) from the other groups using an efficient set of clinical features.

FIG. 18 illustrates in ADEPT-CLIN the top 10 most important clinical characteristics, in order of importance, for differentiating B-SNIP Biotypes. The bar chart at the top illustrates the normalized weights in the adaptive ADEPT algorithm for classifying a case into one group versus all others (BT1 or not, BT2 or not, BT3 or not). The feature names correspond to the items in the specific interview schedule (panss=Positive and Negative Symptom Scale; sfs=Birchwood Social Functioning Scale). The table at the bottom illustrates the means (SDs) of those items with the names associated with their item numbers (e.g., panss_n5=Difficulty in abstract thinking).

FIG. 19 illustrates in ADEPT-CLIN the top 10 most important clinical characteristics, in order of importance, for differentiating DSM diagnoses. The bar chart at the top illustrates the normalized weights in the adaptive ADEPT algorithm for classifying a case into one group versus all others (SZ or not, SAD or not, BDP or not). The feature names correspond to the items in the specific interview schedule (madrs=Montgomery-Asberg; panss-Positive and Negative Symptom Scale; sfs=Birchwood Social Functioning Scale). The table at the bottom illustrates the means (SDs) of those items with the names associated with their item numbers (e.g., madrs_4-Reduced need for sleep).

FIG. 20 illustrates the ADEPT-COG relative importance of all 76 items for differentiating between psychosis Biotypes. PANSS=Positive and Negative Symptom Scale; MADRS-Montgomery-Asberg Depression Rating Scale; Young=Young Mania Rating Scale; SFS=Birchwood Social Functioning Scale. BT1=Biotype-1; BT2-Biotype-2; BT3=Biotype-3.

FIG. 21 illustrates the ADEPT-COG relative importance of all 76 items for differentiating between DSM diagnoses. PANSS=Positive and Negative Symptom Scale; MADRS-Montgomery-Asberg Depression Rating Scale; Young=Young Mania Rating Scale; SFS=Birchwood.

FIG. 22 illustrates the ADEPT-COG sensitivity, specificity, and AUC (area under the curve) of out of-sample tests for differentiating BT1 (left figure), BT2 (middle figure), or BT3 (right figure) from the other groups using an efficient set of cognitive and clinical features.

FIG. 23 illustrates the ADEPT-COG sensitivity, specificity, and AUC (area under the curve) of out of-sample tests for differentiating schizophrenia (left figure), schizoaffective disorder (middle figure), or bipolar disorder with psychosis (right figure) from the other groups using an efficient set of cognitive and clinical features.

FIG. 24 illustrates for ADEPT-COG the top 10 most important characteristics, in order of importance, for differentiating B-SNIP Biotypes. The bar chart at the top illustrates the normalized weights in the adaptive ADEPT-COG algorithm for classifying a case into one group versus all others (BT1 or not, BT2 or not, BT3 or not). The feature names correspond to specific features. The table at the bottom illustrates the means (SDs) of those features.

FIG. 25 illustrates for ADEPT-COG the top 10 most important characteristics, in order of importance, for differentiating DSM diagnoses. The bar chart at the top illustrates the normalized weights in the adaptive ADEPT-COG algorithm for classifying a case into one group versus all others (schizophrenia or not, schizoaffective disorder or not, bipolar disorder with psychosis or not). The feature names correspond to specific features. Madrs=Montgomery-Asberg; pans=Positive and Negative Symptom Scale; sfs=Birchwood Social Functioning Scale). The table at the bottom illustrates the means (SDs) of those features with the names associated with their item numbers (e.g., madrs_4=Reduced need for sleep).

FIG. 26 illustrates an exemplary computing device.

FIG. 27 illustrates the steps involved in implementing aspects of the instant disclosure. Details of the flow chart are described in the text.

Other features of the present embodiments will be apparent from the Detailed Description that follows.

### DETAILED DESCRIPTION OF THE INVENTION

In some aspects, the current disclosure provides improved methods for diagnosis and/or treatment of one or more serious psychiatric conditions.

In some aspects, the current disclosure provides improved methods for diagnosis and/or treatment of psychosis.

In some aspects, the current disclosure provides improved methods for diagnosis and/or treatment of psychosis, by utilizing bio-factors.

In some aspects, the current disclosure provides improved methods for diagnosis and/or treatment of psychosis, by utilizing an adaptive algorithm that that has been trained, on a computer, using a set of clinical information, cognitive information, and/or behavioral information to recognize one or more subtypes of psychosis. Behavioral information can involve data or insights related to observable behaviors of individuals. Clinical information can refer to data collected about a person's mental, emotional, and behavioral health. Cognitive information can refer to data or mental processes related to how individuals acquire, process, store, and/or use information. It involves mental functions that include perception, memory, attention, reasoning, decision-making, problem-solving, language, and/or learning.

### I. Definitions

As used herein, an “adaptive algorithm” refers to an algorithm that operably linked to a computing device and interacts with and/or presents data to an individual based data it processes from the individual. The data that the algorithm presents and/or processes can originate from audio data, visual data, audio-visual data, optical data, electrical data, magnetic data, electromagnetic data, mechanical data, or a combination thereof. An example of an adaptive algorithm includes an adaptive diagnostic algorithm described herein.

As used herein, an “adaptive decision tree” refers to a decision tree model capable of updating its features as it encounters new data, rather than being trained once and left static.

“Bio-factor” refers to a neurobiological variable with numerical values associated with cognitive and/or neurological responses of a subject. The neurobiological variable can be identified from a larger context of neurobiological variables through a dimension-reduction analysis, such as principal component analysis.

As used herein, an “extra-trees classifier” refers to an ensemble learning method that builds several decision trees and combines their predictions.

“Neurobiological,” as relates to diagnosis, refers to diagnosis that uses cognitive and/or neurological responses of a subject.

As used herein, “operably linked” refers to the connection of at least two components in an MRI or other neurobiological data processing system via technology including, but not limited to, integrated circuits, electrical cables, ethernet, internet, intranet, Bluetooth, near field communication, WiFi, or a combination thereof.

As used herein, a “randomized ensemble of classifiers” refers to a technique that combines predictions from multiple individual classifier models to achieve higher accuracy and greater robustness than any single model alone. The “randomized” aspect refers to the various ways in which diversity is introduced among the individual classifiers, making them learn different aspects of the data and thus contributing unique perspectives to the final prediction.

The term “real-time” refers to data transmission to a user interface of a computer-implemented method, system, tool, or device within 1, 2, 3, 4, 5, 10, 15, 20, or no more than 30 minutes after the computer-implemented method, system/tool/device receives input data. The transmitted data can be a neurobiological diagnosis based, in part, on processing input data.

### II. Systems and Methods for Diagnosing Psychosis Subtypes

In the following detailed description of the preferred embodiments, reference is made to the accompanying drawings, which form a part hereof, and within which are shown by way of illustration specific embodiments by which the innovative work may be practiced. It is to be understood that other embodiments may be used, and structural changes may be made without departing from the scope of the innovative work. Electrical, mechanical, logical, and structural changes may be made to the embodiments without departing from the spirit and scope of the present teachings. The following detailed description is therefore not to be taken in a limiting sense, and the scope of the present disclosure is defined by the appended claims and their equivalents.

The present disclosure relates to systems and methods for diagnosing serious psychiatric conditions using an adaptive decision tree algorithm called ADEPT, and the implementation of that diagnosis for selection of treatments for individual patients with an idiopathic psychosis.

In psychosis, knowledge of unique physiology and pathology will improve diagnosis and promote targeting the most effective treatments to the needs of individual patients. There is substantial neurobiological heterogeneity within and overlap between DSM psychosis diagnoses of schizophrenia, schizoaffective disorder, and bipolar disorder with psychosis, the most prominent of the idiopathic psychoses. Treatments, therefore, cannot be targeted to address a patient's specific physiological deviations based on those diagnoses.

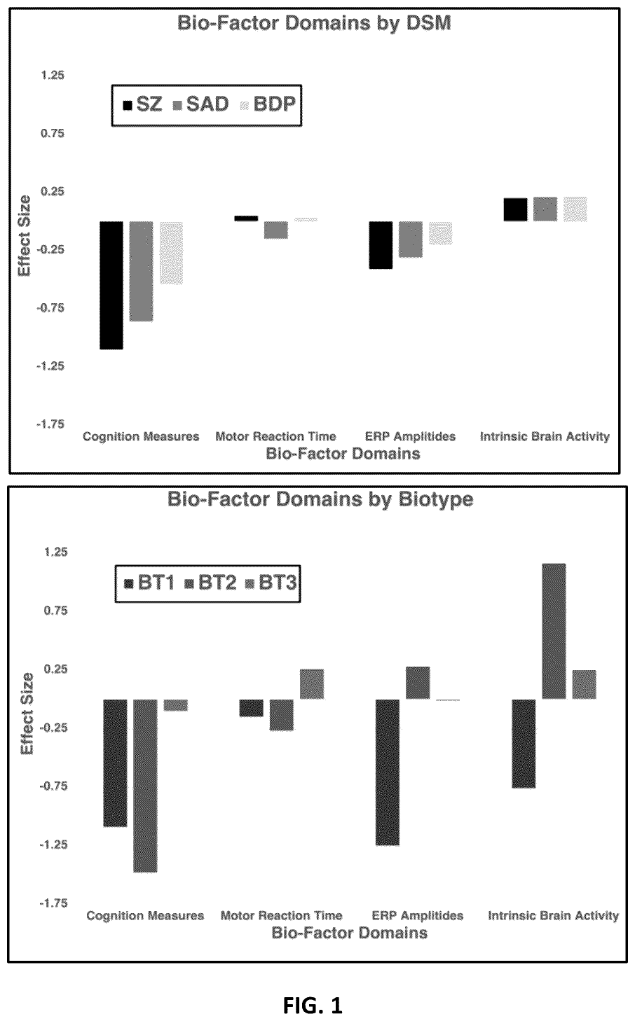

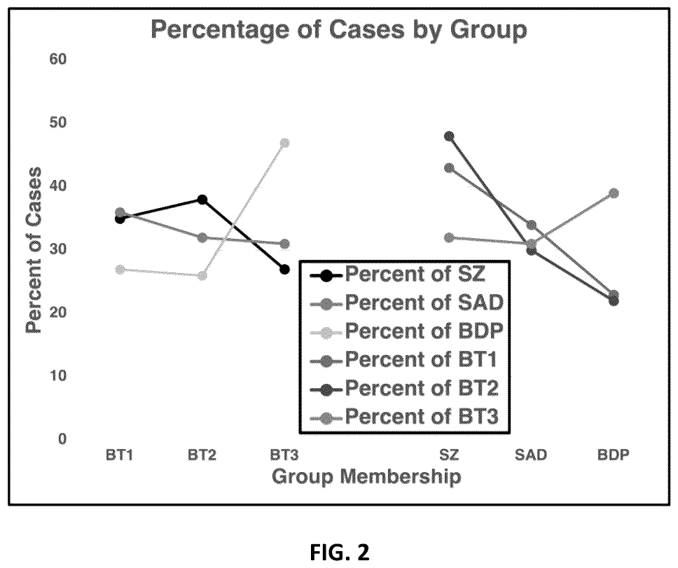

The Bipolar-Schizophrenia Network for Intermediate Phenotypes (B-SNIP), whose members include inventors on this application, improved on this state-of-affairs and developed an approach to enhance standard-of-care. First, as shown in FIG. 1, B-SNIP found no obvious mapping of neurobiological features to DSM schizophrenia, schizoaffective disorder, or bipolar disorder with psychosis, or distinctions between those diagnoses that could be clinically useful. As a result, B-SNIP modified the typical approach to psychosis stratification by creating neurobiologically similar subgroups independent of DSM diagnosis. They discovered neurobiological features characteristic of “B-SNIP psychosis Biotypes” that yield specific treatment targets for patients with an idiopathic psychosis (FIG. 1). The details for obtaining B-SNIP psychosis Biotypes are described in International Patent Application No. PCT/US2025/020864, the contents of which are herein incorporated by reference in their entirety. FIG. 2 illustrates that B-SNIP Biotypes and DSM diagnoses are different approaches to psychosis classification. The following seven sections summarize elements of B-SNIP psychosis Biotypes that make the ADEPT algorithm an important advancement with practical clinical applications. For instance, by using an adaptive algorithm trained as described herein, the computer-implemented method improves sub-type classification of an individual with a subtype of psychosis, by virtue of adapting real-time to the individual's responses to information, and not implementing a one-size-fits-all approach to presenting information to the individual. This also means that information from a large database can be adaptively presented to an individual in for improved and personalized real-time diagnosis of the individual as having a given psychosis sub-type, for subsequent effective treatment prescriptions specific to the individual. Further, by using an adaptive algorithm, rather than a random or rigid presentation of information to an individual, processing of further information can be terminated when an acceptable result has been attained, thereby avoiding wasted cycles of computer processing and/or decrease computational burden.

### (1) Robustness of Biotypes Solution

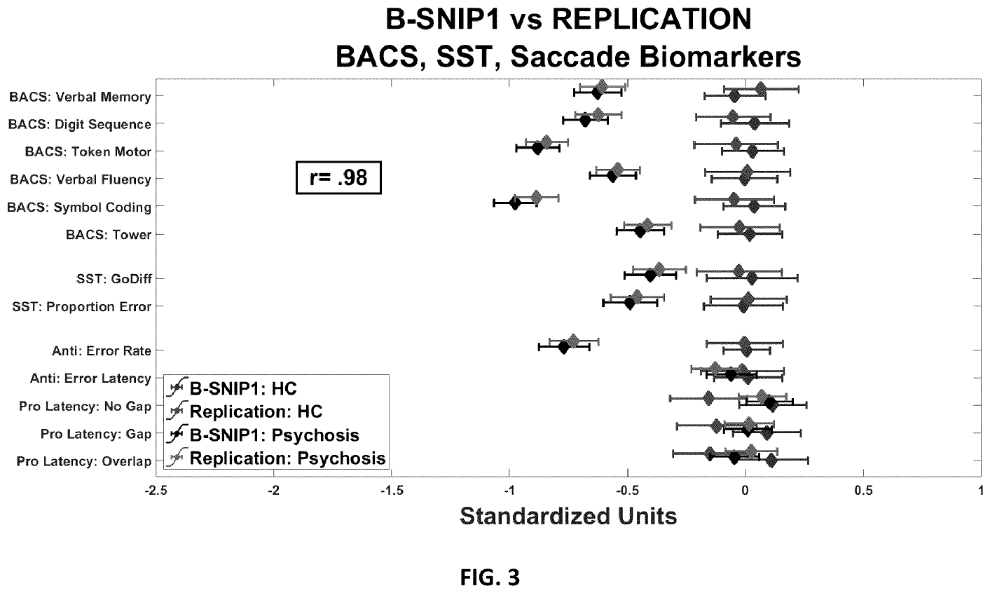

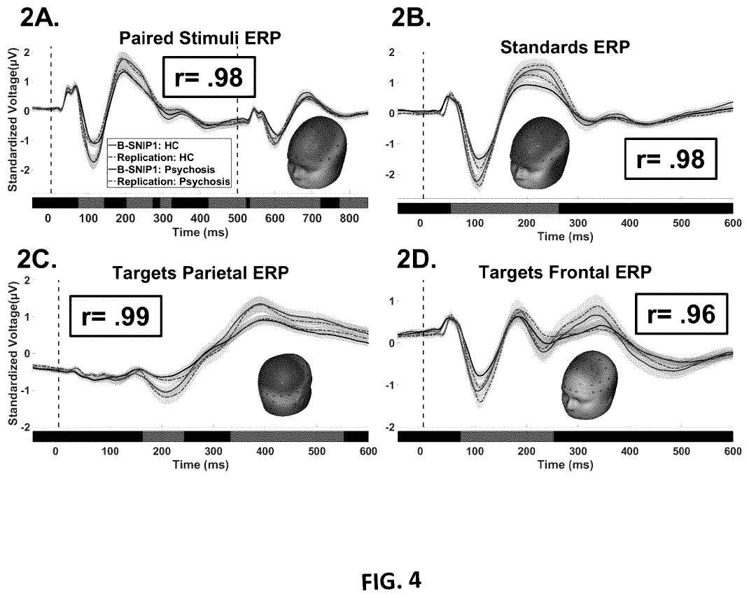

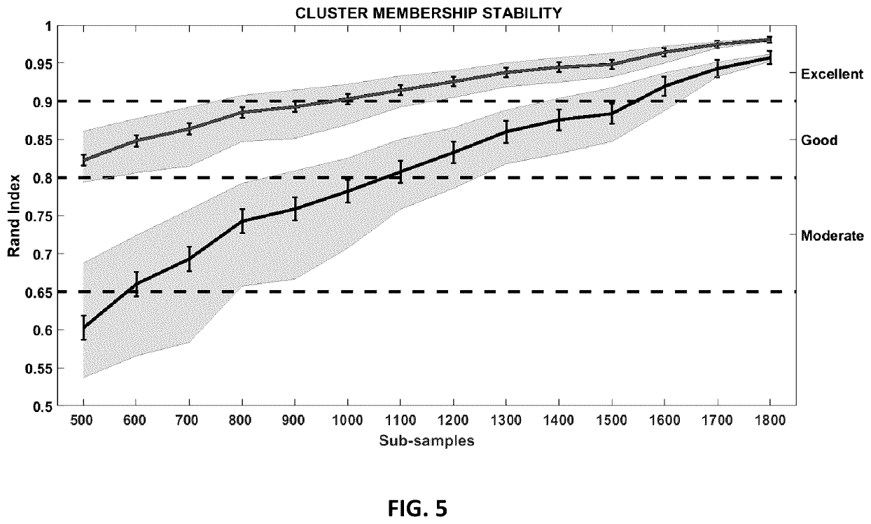

B-SNIP replicated all steps in the biomarker and bio-factor quantification process (see FIG. 3 and FIG. 4), replicated B-SNIP psychosis Biotypes across two samples, and cross-validated B-SNIP psychosis Biotypes between two samples. B-SNIP evaluated the consistency of assigning a patient to their proper group as a function of sample size used to derive the psychosis Biotypes solution. FIG. 5 illustrates the consistency of Biotypes membership using a sub-sampling approach (1000 iterations at each subsample size from 500 to 1800 probands). The figure illustrates two consistency estimates. The first rand index (upper line) illustrates consistency with the full model solution without adjusting for chance assignment. This plot illustrates remarkable consistency of >95% agreement for samples of greater than 1500 observations, excellent agreement of >90% for sample sizes of greater than 900, and good agreement >82% for sample sizes of at least 500. The second rand index (lower line) illustrates consistency adjusting for the probability of a case being assigned by chance to one of the three groups. This conservative metric illustrates remarkable agreement of >95% for samples of greater than 1600, excellent agreement of >90% for samples of greater than 1500, and good agreement of >80% for samples of greater than 1000. These plots illustrate that a combination of B-SNIP laboratory tests, data collection, quantification, and statistical analysis, and sufficiently large samples sizes, are necessary to obtain a stable and clinically implementable biomarker-based diagnostic algorithm for idiopathic psychosis. These analyses support the utility of the ADEPT algorithm.

### (2) Magnitude of Biotypes Neurobiological Differences

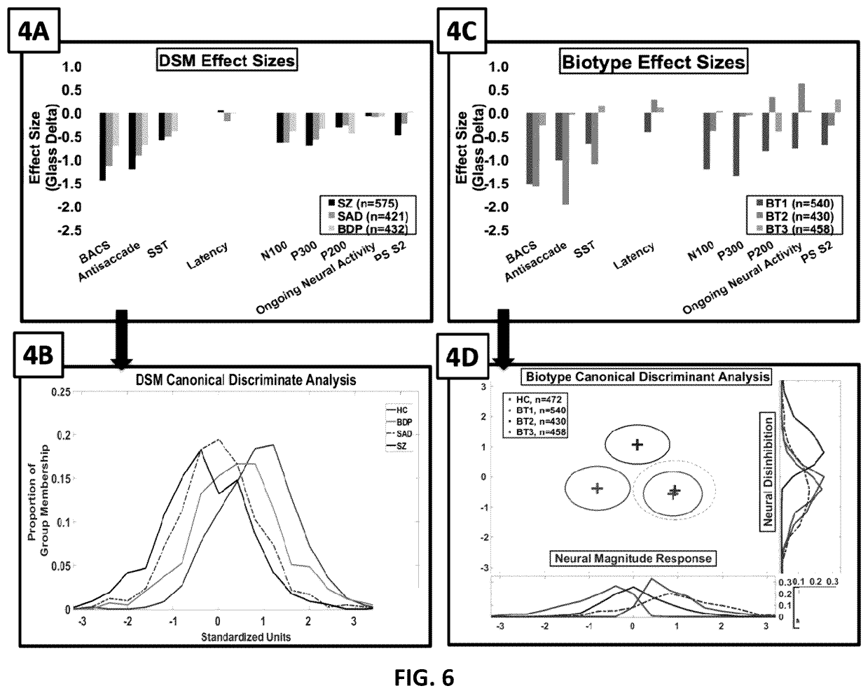

FIG. 6 illustrates a more detailed look at bio-factors plotted by Biotypes and DSM diagnosis for the psychosis patients. All bio-factors differentiate psychosis Biotypes (Holm-Bonferroni adjusted significance, F's=20.21 to 703.81, p's<0.001). The bio-factor patterns for psychosis groups in comparison to healthy performance are robust because of novel, reliable, and comprehensive analytical methods. BT1 patients have low cognitive performance and low neural response magnitudes. BT2 patients have low cognitive performance, the poorest inhibition, and accentuated intrinsic brain activity. BT3 patients are reasonably normal across most bio-factors but mildly deviant on measures of stimulus salience. FIG. 6 illustrates that Biotypes are distributed in multi-dimensional space, that space is defined by implementable treatment targets; DSM diagnoses have a modest and largely overlapping uni-dimensional distribution with no obvious treatment targets.

### (3) Validation of Psychosis Biotype-Defining Electrophysiology

Differentiating features of BT1 and BT2 are low neural response to salient stimuli (BT1) and excessive intrinsic and background brain activity (BT2). Both BT1 and BT2 deviations result in poor signal-to-noise (BT1 because of a small numerator and BT2 because of a large denominator). B-SNIP used an auditory steady-state paradigm to directly probe these features using a laboratory task that is not part of Biotypes' creation.

In steady-state paradigms, stimuli are modulated at known frequencies (e.g., 40-Hz, an event every 25-ms) for an extended time (e.g., 1500-ms). Neurons tuned to those oscillations resonate at the stimulation frequency. There is a known input (40-Hz signal) and an expected output (40-Hz oscillations in the EEG). This one paradigm allows for simultaneous evaluation of the “neural dysregulation” characteristic of BT1 and the “neural vigor” characteristic of BT2. If the neurophysiological model that supports specific diagnosis and treatment targeting is correct, there will be a double dissociation of BT1 and BT2 defining physiologies.

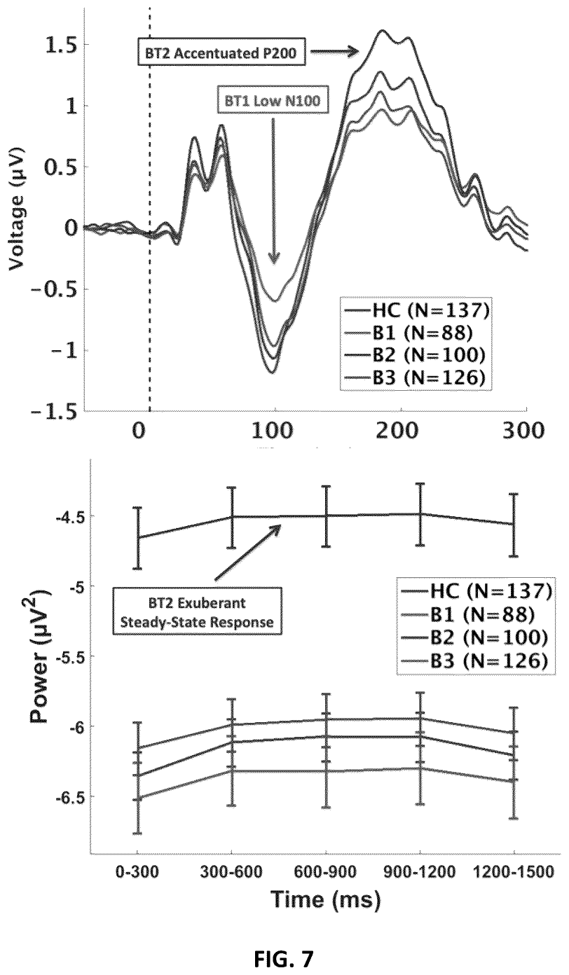

The upper part of FIG. 7 illustrates ERPs to stimuli onset in the steady state paradigm by group. Only BT1 have reduced N100 ERP magnitude, part of BT1's unique defining feature of low neural vigor. BT2 have normal magnitude N100 ERP, but accentuated P200 ERP, which coincides with the beginning of the auditory steady state response (seen as “divots” in the ERP). Following the P200, the lower part of FIG. 7 illustrates power at the driving frequencies during ongoing steady-state stimulation. BT2 are the only group that is accentuated on this characteristic of exaggerated neural responding, one of the defining features of neural vigor. These physiology differences yield specific etiological and treatment targets that are not available with any other psychosis classification system and support the utility of ADEPT.

### (4) Structural Brain Imaging Validation of Psychosis Biotypes

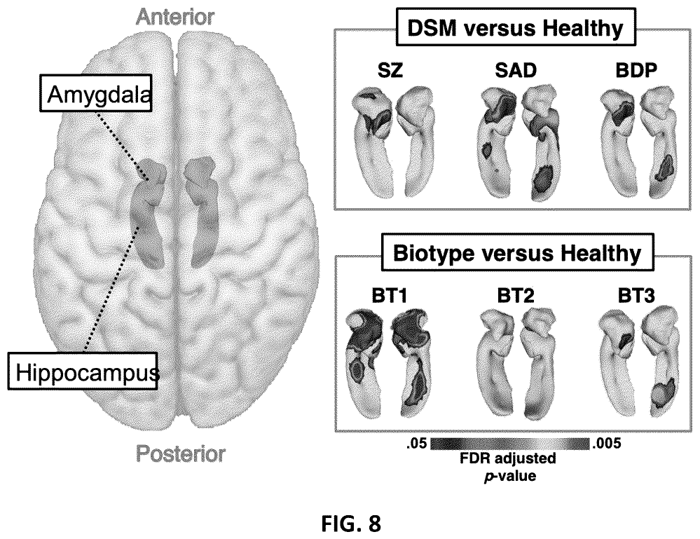

Biotypes are defined by cognition and electrophysiology, but they capture unique deviations across levels of analysis, including brain morphometry. There are associations between the hippocampal complex and a subset of psychosis cases. How to capture that subset of psychosis cases has been ambiguous. B-SNIP psychosis Biotypes resolve that ambiguity with surprisingly accuracy. B-SNIP assessed hippocampal and amygdala volume and shape deformities in 475 psychosis cases and 315 healthy subjects. Volume and shape outcomes were highly similar; FIG. 8 illustrates the outcomes for shape (DSM at top of FIG. 8, and Biotypes at bottom of FIG. 8): (i) DSM groups did not differ on hippocampal and amygdala volume or shape; (ii) BT2, BT3, and healthy groups are indistinguishable on hippocampal volume and shape; (iii) BT1 significantly differ from BT2, BT3, and healthy groups (average effect size=0.42). These outcomes illustrate three important points: (i) DSM groups do not capture this important differentiating neuroanatomical feature; (ii) sorting idiopathic psychosis patients by B-SNIP Biotypes illustrates that hippocampal complex deviations are largely restricted to BT1; (iii) defining cases by neurobiological homology supports organization of neuropathology, and therefore treatment targeting, at multiple levels of analysis. These outcomes support the importance of the ADEPT algorithm.

### (5) Structural Brain Imaging Diagnosis of Psychosis Biotypes

B-SNIP illustrated that a supervised machine learning approach applied to voxel-by-voxel brain gray matter densities classifies B-SNIP psychosis Biotype-1 (BT1). T1-weighted structural images were acquired using 3T MRI. The analysis pipeline incorporated the DARTEL high-dimensional nonlinear inter-subject registration tool. Gray matter densities were extracted from the segmented and modulated gray matter images using a gray matter mask. A total of 371,243 gray matter density features were used in the machine learning analyses.

Machine learning analyses extracted patterns of gray matter densities that reliably differentiate BT1 from other groups. B-SNIP used a repeated train-then-test split approach with 1000 iterations. For each iteration, a randomly selected subset of the data trained the classification model, and the held-out data tested the model. Three binary classification models were trained to discriminate one of the three Biotypes versus healthy persons. The trained models were applied to every case in the held-out test groups. Classifier accuracy was computed using a balanced accuracy metric, the unweighted average of each group's classification accuracy, or the average of the sensitivity and specific of the classifier.

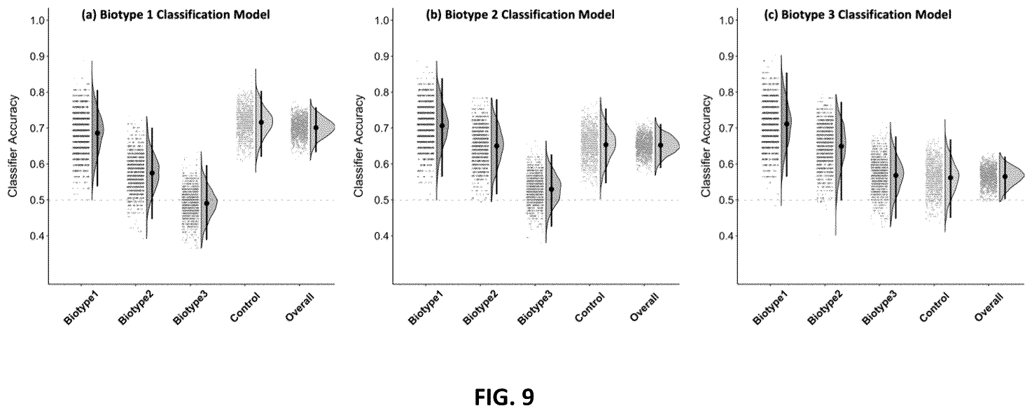

The results for the three Biotypes classification models are illustrated in FIG. 9. For BT1, overall classification accuracy was significantly above chance. Classification accuracies were also significantly above chance for healthy persons. Importantly, the model did not classify either BT2 or BT3 cases as belonging to the BT1 group at rates above chance. This pattern illustrates specificity of this gray matter density diagnostic algorithm to BT1.

Classification accuracy of the BT2 model was driven by gray matter density features common to both BT1 and BT2, and not features specific to BT2. Lastly, neither the separate classification accuracies for BT3 nor for healthy persons exceeded chance. The BT3 model did not show specificity as BT1 cases were misclassified as belonging to the BT3 group at rates greater than chance. BT2 cases, however, were not misclassified as BT3 above chance. These outcomes illustrate that classification performance of the BT3 model was driven by gray matter density features common to all Biotypes and healthy persons.

This gray matter algorithm has surprising specificity for diagnosing BT1. Despite similarity in cognitive performance between BT1 and BT2, gray matter densities only diagnose BT1. This illustrates that the same superficial features can have different causes and neurobiological correlates. This is why DSM diagnoses, which are only based on superficial features, do not yield specific neurobiological treatment targets. DSM diagnoses of schizophrenia and bipolar disorder were not classified above chance using the same MRI approach. Notably, the classifier algorithm was trained on whole-brain gray matter density features that were not used to derive Biotypes. Ergo, this gray matter density machine learning algorithm provides independent validation for B-SNIP psychosis Biotype-1 (BT1). These outcomes support the utility of the ADEPT algorithm.

### (6) Sensory Training Validation of Psychosis Biotypes

The physiological differences between B-SNIP psychosis Biotypes offer specific treatment targets not provided by any other approach for diagnosing idiopathic psychosis patients. The only way to determine improved efficacy for treatment selection is to directly compare available approaches.

BT1 and BT2 patients have poor signal-to-noise, the former due to deficient neural responses to salient stimuli and the latter due to accentuated intrinsic or background brain activity. Signal-to-noise is the difference between strength of neural response to a specific stimulus (like an auditory or visual event in the environment) and the background level of ongoing neural activity in the brain. The bigger this difference, the better a person can accurately process the stimulus of interest. Both BT1 and BT2 patients have low signal-to-noise and need treatment to correct this deviation. The means to achieve that end, however, differ by BT group. One approach involves sensory training to specifically enhance ERP magnitudes in BT1 because they need stronger neural response to the stimulus to improve their signal-to-noise. Such approaches have been tried with schizophrenia cases, who are a mix of different B-SNIP psychosis Biotypes. Those studies have shown at best only modest success.

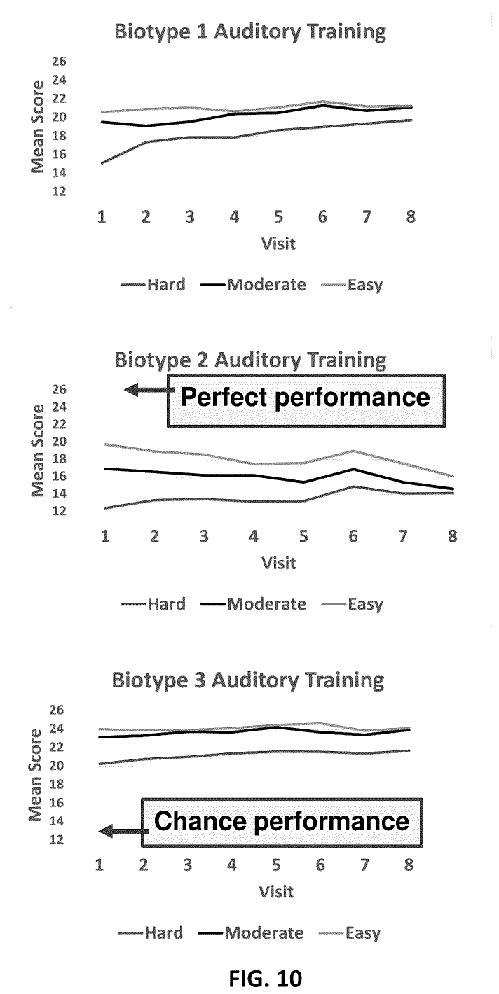

Patients complete three weeks of sensory training targeting the underlying problem of BT1. FIG. 10 illustrates the training outcomes. The x-axis illustrates eight training days (4 training sessions per day). Behaviorally on signal discrimination, BT1s improve on all conditions, BT2 have the worst overall performance, and across all conditions they deteriorate over time, and BT3 have the best performance overall, but show no significant improvement over training. These outcomes illustrate that B-SNIP psychosis Biotypes yield implementable and meaningful treatment targets that are not derivable from any other available approach to idiopathic psychosis diagnosis.

B-SNIP illustrates that only BT1 benefit from a sensory training treatment aimed at enhancing ERP magnitudes because they are the only group with that specific deviation. BT2 have neural dysregulation in association with stimulus processing, so participation in the same paradigm that improves BT1 worsens BT2 because processing the stimuli degrades BT2 signal-to-noise ratios even further. This is an iatrogenic effect on BT2 of a commonly employed sensory training procedure, but this effect is only identifiable by implementing B-SNIP psychosis Biotypes. This same differential treatment efficacy is not evident when using DSM psychosis diagnoses. This outcome supports the importance of the ADEPT algorithm.

### (7) Selecting Patients for Clozapine Treatment Using Psychosis Biotypes

Clozapine is the most effective antipsychotic drug, but it is underused because of side effects concerns and sometimes complex administration. If there was a means to identify responsive cases in advance, clozapine could be used more decisively. Clozapine increases alpha and theta electroencephalography power in a resting state and modifies signal-to-noise ratios in some patients. How those effects relate to psychosis treatment is unclear using DSM diagnosis but is clarified by implementing B-SNIP psychosis Biotypes.

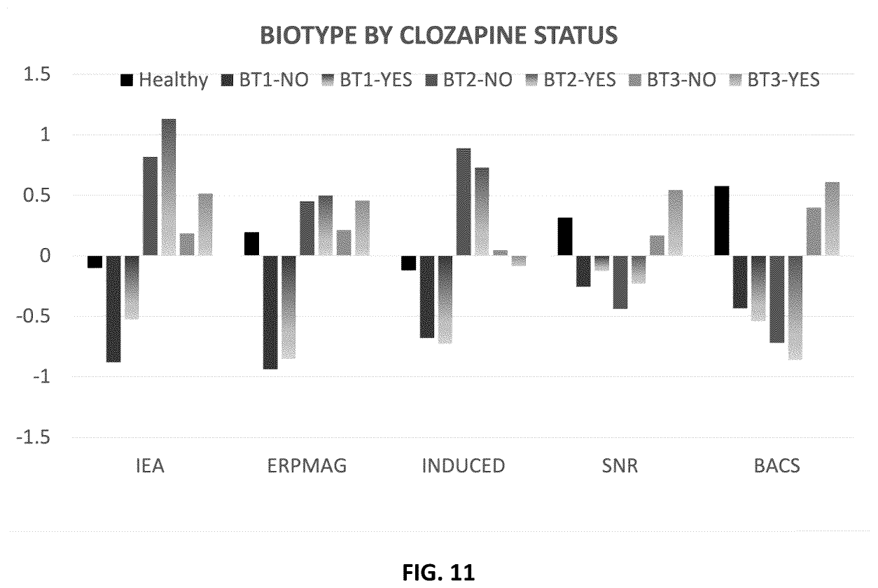

B-SNIP has cases off and on clozapine. FIG. 11 illustrates the relationships between clozapine status and relevant B-SNIP bio-factors and biomarkers. The largest number of patients are not taking clozapine (n=1763, roughly evenly spread across the three Biotypes). A smaller number of patients are taking clozapine (n=140, roughly evenly spread across the three Biotypes).

There are five main conclusions from the bar charts in FIG. 11: (i) Being on clozapine is associated with increased intrinsic EEG activity (IEA) regardless of psychosis group. Nevertheless, being on clozapine is associated with a closer to normal level of IEA among BT1 patients but more deviant IEA among BT2 and BT3 patients. If adjusting level of IEA is related to treatment success, BT1 patients are the ones who should be targeted. (ii) ERP magnitudes are significantly larger only among BT3 patients on clozapine versus not on clozapine. (iii) Level of induced EEG activity, which is brain activity during stimulus processing that is not part of the ERP response, is modestly lower among all patients on clozapine versus not on clozapine. Level of induced EEG activity is closer to normal among BT3 patients on clozapine versus not on clozapine. (iv) Level of signal-to-noise (ERP magnitude versus induced EEG activity) is modestly larger in every psychosis group on clozapine, but only significantly larger among BT3 patients on clozapine versus not on clozapine. If adjusting signal-to-noise is related to treatment success, BT3 patients are the ones who should be targeted. BT3 is also the only group in the healthy range on the signal-to-noise measure. (v) Both BT1 and BT2 patients on clozapine have worse general cognitive performance when on clozapine versus not on clozapine. This could be because clinicians treat the most cognitively compromised patients with clozapine. In comparison, however, this simple interpretation is not consistent with the fact that BT3 patients on clozapine have better general cognitive performance (in the healthy range) than those not taking clozapine. Ergo, B-SNIP psychosis Biotypes yield specific treatment targets for clozapine, the most effective antipsychotic medication. There is no such predictive ability provided by any other approach to psychosis diagnosis. This outcome supports the importance of the ADEPT algorithm.

### (A) The ADEPT-CLINICAL (ADEPT-CLIN) Decision Tree Algorithm

Careful evaluation of a patient's history and clinical presentation is the bedrock of medicine. Many medical diagnoses begin when a perceptive clinician notices a constellation of signs and symptoms across many such assessments of multiple patients. The related occurrence of clinical features, called a syndrome, may indicate a disease or group of diseases, but there is no guarantee. There must be proof of differentiable pathology to clinical presentation links to support the identification of a disease. An unverified assumption within psychiatry is that its clinical diagnoses harbor different diseases (i.e., specific pathophysiologies linking to clinical presentation) with similar clinical pictures but different etiologies and pathophysiologies.

A problem for psychiatry is the direction of evaluation: psychiatry hopes to use clinical features to derive specific pathologies, which is different from starting with the pathology and identifying the linked clinical features. A complication for the clinical features to pathology approach is the numerous possible pathologies that can yield similar clinical presentations. Hopefully, though, the neurobiological origins of psychiatric syndromes can be parsed via laboratory examination to stratify patients and design pathophysiology-specific treatments. This innovative work details a method for identifying neurobiologically specific psychosis subgroups (Biotypes) using readily available clinical information alone.

Belief in separating clinically defined psychiatric syndromes into distinct diseases anchored Robins and Guze's classical approach. However, it seems unlikely diseases can be carved from current clinical psychosis categories, like those in the Diagnostic and Statistical Manual (DSM) classification. Some psychiatric professionals advocate for patience with contemporary psychiatric diagnoses, hoping that a gradual iterative process will yield basic etiological understanding. There are reasons for skepticism that such an approach will succeed in some areas of psychiatry, like for psychosis.

Contemporary DSM diagnostic criteria derive from committee consensus. Through no fault of well-intentioned committees, group decision-making is prone to systematic errors and cognitive biases, which can distort committee outcomes. For instance, current psychosis diagnostic criteria may have wandered from the original syndromes they were meant to operationalize, and perhaps are inefficient tools for deriving specific etiologies. The great Paul Meehl warned how similar conceptual and meaning drift, in the spirit of psychometric efficiency or explanatory expediency, can markedly and hopelessly morph the original phenomenon of study. Even a thorough clinical evaluation will be suboptimal for etiological and treatment investigation if it fails to capture a valid diagnostic entity, meaning one with ties to specific etiological factors.

Despite the importance of clinical evaluation, it is also proposed that medical disciplines must abandon exclusive reliance on clinical definitions and incorporate additional data like laboratory tests to cleave individual diseases from a heterogeneous clinical stew. Indeed, Samuel Guze advocated for a shift from clinical features alone to inclusion of laboratory tests in psychiatric diagnoses. Psychosis diagnostics have not advanced along such lines.

A possible difficulty for incorporating laboratory tests in DSM-type psychosis categories is illustrated by the B-SNIP program. B-SNIP demonstrated (n>700 psychosis cases) and replicated (n>700 psychosis cases) that DSM psychosis diagnoses don't capture neurobiologically distinct entities. Across most measures, psychosis diagnoses describe a neurobiological continuum (schizophrenia<schizoaffective disorder<bipolar disorder<healthy persons), with considerable group overlap.

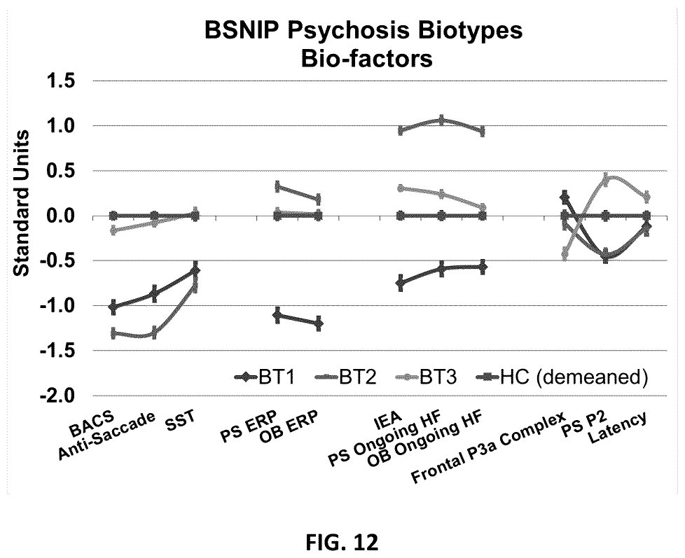

So current clinical psychosis diagnoses fail to approximate distinct neurobiological entities with pathology-specific treatment targets. Consequently, over 40 years of patience with DSM and ICD-type diagnostic categories as gold standards has yielded few diagnosis-specific treatment advances for persons with idiopathic psychosis. As an alternative, B-SNIP searched for neurobiological similarity within idiopathic psychosis cases, regardless of DSM diagnosis. B-SNIP used variance across multiple psychosis-relevant laboratory measures and numerical taxonomy statistics to form comparable clusters of individuals. A fine-grained outcome of this approach is illustrated in FIG. 12. B-SNIP identified, replicated, cross-validated, and externally validated transdiagnostic subgroups called psychosis Biotypes. Biotype-1 (BT1) and Biotype-2 (BT2) share marked cognitive performance deficits. Biotype-1's defining feature, however, is weak neural response across multiple neurophysiological measures. Alternatively, Biotype-2's defining feature is excessive intrinsic neural brain activity. Biotype-3 (BT3) cases are close to normal across many cognitive, psychomotor, and neurophysiological functions, but have specific deviations on tests measuring stimulus salience.

Stratification of psychosis cases by neurobiology may facilitate the search for specific etiology and improve treatment targeting. But clinical evaluation through observation and interview is always the first step in medical diagnosis, with clinical presentation advising the selection of laboratory tests. Even though B-SNIP Biotypes were formed using laboratory measures, the defining neurobiological features of psychosis Biotypes may still map to constellations of signs and symptoms.

The present section provides the first iteration of an adaptive algorithm, ADEPT-CLIN, that generates B-SNIP psychosis Biotype diagnoses. This innovative work illustrates that (i) neurobiologically homologous psychosis subgroups (Biotypes) have clinical features that enlighten their differential identification, and (ii) unexpectedly, those features do not match or simply recapitulate clinical characteristics that differentiate DSM psychosis diagnoses. This innovative work illustrates that clinical characteristics differentiate B-SNIP Biotypes, so like in any other branch of medicine clinicians can use those features to start the diagnosis of neurobiological subgroups. This innovative work will improve etiological investigations and treatment applications of Biotypes compared to alternatives and improve standard-of-care for all psychosis patients.

### Methods and Analyses

In the B-SNIP database, there were 1907 psychosis cases with clinical and laboratory data for inclusion in this innovative work. B-SNIP recruitment sites were in Athens GA (University of Georgia), Baltimore MD (Maryland Psychiatric Research Center), Boston MA (Beth Israel Deaconess Medical Center), Chicago IL (University of Illinois-Chicago and University of Chicago), Dallas TX (UT Southwestern Medical Center), Detroit MI (Wayne State University), and Hartford CT (Institute of Living). See Table 1 for demographic information by Biotype, including participant and family-of-origin socioeconomic status from the Hollingshead Two-Factor Index, and Table 2 for this same information by DSM diagnosis. All subject recruitments, interviews, and laboratory data collections occurred at B-SNIP locations. Cases were drawn from academic and community mental health centers, small towns with large universities, large cities, inner cities, rural regions, affluent and less affluent areas. B-SNIP recruited a research sample, not an epidemiological sample; nonetheless, the large study numbers and broad geographical recruitment foster generalizability of the outcomes across the range of early onset through midcourse to chronic idiopathic psychosis.

TABLE 1
Demographic characteristics by Biotype

| | Overall | Biotype-1 | Biotype-2 | Biotype-3 | Healthy |
|---|---|---|---|---|---|
| | N = | N = | N = | N = | N = |
| Characteristic | 1907 | 630 | 631 | 646 | 895 |
| Mean age (SD) | 38 (12) | 38 (13) | 39 (12) | 36 (12) | 35 (12) |
| Sex: | | | | | |
| Male | 51% | 57% | 44% | 51% | 43% |
| Female | 49% | 43% | 56% | 49% | 57% |
| Ethnicity | | | | | |
| Not Hispanic | 88% | 86% | 88% | 90% | 88% |
| Hispanic | 12% | 14% | 12% | 10% | 12% |
| Race | | | | | |
| Black | 39% | 50% | 42% | 24% | 29% |
| Amer. Indian | 0.4% | 0.8% | 0.2% | 0.3% | 0.2% |
| Asian | 3.0% | 3.4% | 1.9% | 3.6% | 8.3% |
| White | 50% | 39% | 47% | 64% | 56% |
| Multiracial | 5.1% | 4.6% | 5.6% | 5.0% | 3.6% |
| Other | 2.8% | 2.6% | 2.9% | 3.0% | 2.6% |
| Mean GAF (SD) | 53 (13) | 52 (12) | 51 (12) | 56 (14) | 85 (7) |
| Mean Participant | 47 (15) | 50 (14) | 49 (14) | 43 (15) | 35 (14) |
| SES (SD) | | | | | |
| Mean Family | 42 (16) | 44 (16) | 44 (16) | 38 (16) | 37 (15) |
| SES (SD) | | | | | |

TABLE 2
Demographic characteristics by DSM Diagnosis

| | | | | Bipolar |
|---|---|---|---|---|
| | Overall | Schizophrenia | Schizoaffective | Psychosis |
| Characteristic | N = 1907 | N = 783 | N = 582 | N = 542 |
| Mean age (SD) | 38 (12) | 38 (13) | 39 (12) | 36 (12) |
| Sex: | | | | |
| Male | 51% | 63% | 44% | 40% |
| Female | 49% | 37% | 56% | 60% |
| Ethnicity | | | | |
| Not Hispanic | 88% | 90% | 86% | 88% |
| Hispanic | 12% | 10% | 14% | 12% |
| Race | | | | |
| Black | 39% | 50% | 40% | 21% |
| Amer. Indian | 0.4% | 0.4% | 0.3% | 0.6% |
| Asian | 3.0% | 4.2% | 2.1% | 2.0% |
| White | 50% | 39% | 47% | 71% |
| Multiracial | 5.1% | 4.4% | 7.6% | 3.3% |
| Other | 2.8% | 2.8% | 3.1% | 2.4% |
| Mean GAF | 53 (13) | 50 (12) | 52 (12) | 59 (13) |
| (SD) | | | | |
| Mean | 47 (15) | 51 (14) | 48 (14) | 42 (15) |
| Participant SES | | | | |
| (SD) | | | | |
| Mean Family | 42 (16) | 43 (16) | 43 (16) | 38 (16) |
| SES (SD) | | | | |

### Clinical Evaluation

Clinically stable outpatients were administered the Structured Clinical Interview for DSM diagnosis. Psychosis cases were limited to schizophrenia, schizoaffective disorder, and bipolar I disorder with psychosis because these are the idiopathic diagnoses with the highest prevalence in most settings. Cases were rated on the Birchwood Social Functioning (SFS), Montgomery-Asberg Depression Rating (MADRS), Positive and Negative Syndrome (PANSS), and Young Mania Rating (YMRS) scales.

The extensive clinical information on every individual was reviewed in a best-estimate diagnostic meeting with at least two experienced research clinicians to establish the consensus diagnosis. Cross-site diagnostic conference calls were carried out monthly, chaired by two senior primary investigators, and attended by the 2-4 trained clinical assessors at each site. From study start, there were face-to-face and virtual training sessions for all raters, with a requirement for reliability above 0.85. Table 3 shows the average clinical information by Biotype and Table 4 shows this same information by DSM diagnosis.

TABLE 3
Clinical characteristics- mean (SD) - by Biotype

| | Overall | | Biotype-1 | | Biotype-2 | | Biotype-3 | |
|---|---|---|---|---|---|---|---|---|
| Scale | N = 1907 | | N = 630 | | N = 631 | | N = 646 | |
| Birchwood | 123 | (25) | 121 | (24) | 119 | (24) | 129 | (24) |
| Social | | | | | | | | |
| Functioning | | | | | | | | |
| Scale | | | | | | | | |
| Positive- | 62 | (19) | 63 | (19) | 66 | (19) | 58 | (19) |
| Negative | | | | | | | | |
| Syndrome | | | | | | | | |
| Scale | | | | | | | | |
| Montgomery- | 11 | (10) | 11 | (10) | 11 | (10) | 11 | (10) |
| Asberg | | | | | | | | |
| Depression | | | | | | | | |
| Scale | | | | | | | | |
| Young Mania | 8 | (7) | 8 | (7) | 8 | (8) | 7 | (7) |

Scale

TABLE 4
Clinical characteristics- mean (SD) - by DSM diagnosis

| | | | | | Bipolar | |
|---|---|---|---|---|---|---|
| | Schizophrenia | | Schizoaffective | | Psychosis | |
| Scale | N = 783 | | N = 582 | | N = 542 | |
| Birchwood Social | 119 | (23) | 119 | (25) | 133 | (23) |
| Functioning Scale | | | | | | |
| Positive-Negative | 65 | (19) | 66 | (19) | 54 | (17) |
| Syndrome Scale | | | | | | |
| Montgomery-Asberg | 9 | (8) | 14 | (10) | 12 | (11) |
| Depression Scale | | | | | | |
| Young Mania Scale | 7 | (7) | 10 | (8) | 7 | (8) |

### Biotype Evaluations

Participants completed comprehensive laboratory evaluations within a few weeks of their clinical assessments. Details of biomarker quantification and biotyping procedures are in multiple previous publications and in International Patent Application No. PCT/US2025/020864, the contents of which are herein incorporated by reference in their entirety.

The seven laboratory measures used for Biotypes creation were the Brief Assessment of Cognition in Schizophrenia (BACS), Stop-Signal Task (SST), pro- and anti-saccade tasks (saccades), auditory paired stimuli and oddball tasks (ERPs), and the 9-10 second inter-pair interval of the paired stimuli task (intrinsic EEG activity or IEA). Within each laboratory measurement class (BACS, SST, saccades, ERP, IEA), principal component analysis reduced multiple variables within that class to an efficient and smaller variable set. This was done for two main reasons. First, to estimate the true value on any theoretical construct, multiple independent measures are better than any single variable. Cognitive performance and personality tests, for instance, use many questions to estimate the trait of interest. Likewise, for example, neural response to stimulus salience is better estimated by many ERP measures than by a single voltage from a single sensor at one time point. Second, reducing the redundancy of measurements increases the accuracy of numerical taxonomy.

This data reduction process produced variables called bio-factors, which were used in numerical taxonomy. Every subcomponent of the above procedure replicated in two independent samples containing >700 psychosis cases and >200 healthy participants. The numerical taxonomy outcome cross-validated, and multiple other comparisons support the neurobiological validity of B-SNIP psychosis Biotypes. FIG. 12 shows detailed effect sizes of bio-factors for the same subjects plotted by either their Biotype or DSM diagnosis.

### Statistical Methods

B-SNIP Biotypes are multivariate biomarker-derived subgroups, with a range of cases from 15 to 62 years of age at all stages of illness. For creating neurobiological subgroups, B-SNIP used laboratory measures at an intermediate level of analysis in the causal chain (between molecular and clinical levels). This innovative work illustrates that B-SNIP Biotypes have distinctive clinical characteristics that can be used for diagnosis, despite being defined by neurobiological characteristics. Those clinical characteristics differ dramatically from those used to generate DSM psychosis diagnoses. This finding unexpectedly illustrates that previous work on psychosis using clinical characteristics tied to DSM diagnoses are not relevant to the study of B-SNIP psychosis Biotypes. The clinical features came from the seven subscales of the Birchwood, 10 items of the MADRS, 30 items of the PANSS, and 11 items of the YMRS, for a total of 58 clinical features.

This innovative work identifies the important clinical features of psychosis Biotypes using decision tree models. One feature of the innovative work is that it reduces patient and clinician burden by reducing the number of features necessary for categorization while maintaining high classification accuracy. An important distinction of the innovative work is that it constrains the number of features used in the decision tree. Algorithms can be built from a relatively large set of items (i.e., the 58 clinical features). By using decision trees or ensembles of decision trees (i.e., a random forest), the number of estimators (trees) in each sample and the maximum depth (number of items, or features, assessed) of each tree can be constrained.

Constraints incur a performance cost. It is known that ensembles and their related architectures can produce significant improvement over classical decision trees by effectively reducing the variance (in bagged ensembles or in random forests) or the bias (in boosted estimators) components of the test error. In the innovative work disclosed herein, an ensemble was built with a limited number of estimators, each of which have a constrained depth. The result is a weighted combination of the component estimators, using weights developed from the whole sample and the all 58-clinical feature calibration data.

The “extra-trees” algorithm of this innovative work maximizes area-under-the-curve (AUC) for the constraint described above, outperforming other related classifiers. The extra-trees method (“extremely randomized trees”) drops the random forest idea of using bootstrap copies of the learning sample (thus reducing variance), and instead selects a cut-point at random, which leads to increased accuracy (due to smoothing) and decreased computational burden. To provide out-of-sample validation, the inventors created an empirical distribution for AUC, where each individual replicate is generated from a random split of the dataset into training and validation subsets. This approach is like “leave-p out cross-validation,” except a dataset fraction is specified instead of a fixed p (validation proportion=0.5, training proportion=0.5). The process is repeated until there is no significant change in the AUC distribution under a Kolmogorov-Smirnov test. Since this split can be modeled as a random draw from the complete dataset, confidence bounds for AUC are computed directly from percentiles of the empirical distribution.

In the present context, the inventors have three groups, BT1, BT2, and BT3, which is a multi-class identification problem with three Biotypes. The inventors used the 58 clinical features at the beginning to develop a low-burden adaptive classifier that can be used by non-B-SNIP sites to derive a Biotype classification based on clinical information alone. The inventors computed a one-vs-all extremely randomized ensemble of decision trees for each of the three Biotypes. While the out-sample AUCs obtained for each individual Biotype may be high, the end-to-end performance for the classifications is lower. In constructing these trees, the inventors investigated enforcing a maximum allowable depth. For example, setting a maximum depth of 10, with 2 distinct estimators in the “forest”, imply that at most 20 features are used to decide the group membership of any given case. Depth means the number of choice points, e.g., if the individual says “yes” to a question, go to one set of queries, otherwise go to a different set of queries. This is an adaptive algorithm, so administered items are determined based on the next most useful bit of information given the response to previous information. The average number of features used for the overall classification of a case into one of the three Biotypes typically can be smaller than the maximum allowable depth. The outcome of this procedure is called Algorithmic Diagnostics for Efficient Prescription of Treatments-Clinical Version (ADEPT-CLIN) and is illustrated below.

### Results

### (i) Overall ADEPT-CLIN Accuracy as a Function of Items

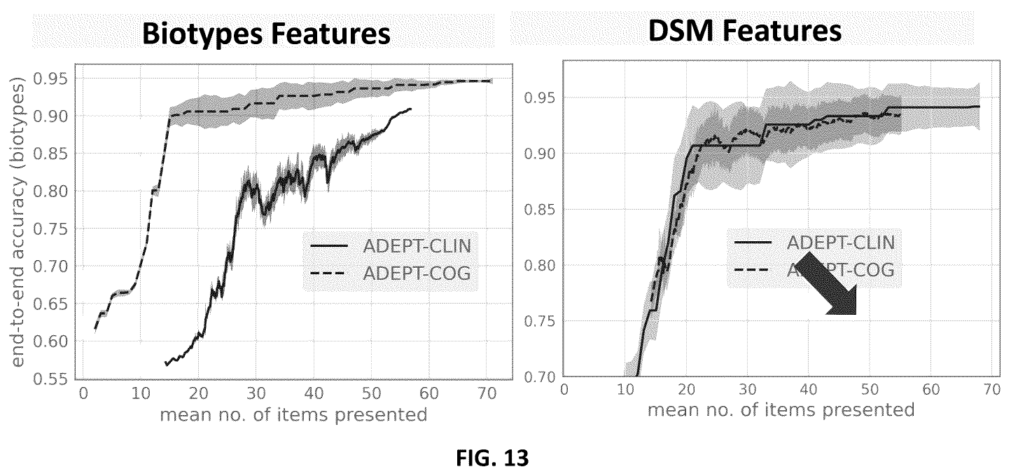

First, the inventors evaluated the overall classification accuracy of the model as a function of the number of items included in the Biotypes classification problem. FIG. 13 shows mean accuracy (with 99% confidence intervals) for classifying a case into one of the three Biotypes as a function of the number of items used (the 58 clinical features). Overall accuracy of correctly assigning an individual case to their Biotype membership peaks at 91% correct classification with 57 items presented on average. In comparison, note in FIG. 13 there is a local maximum in mean out-of-sample classification accuracy of 81% with 28 items presented, which suggests a more efficient clinical evaluation can achieve successful Biotype classification. FIG. 13 also shows this same information for DSM diagnoses.

### (ii) Accuracy of Individual Biotype Assignment

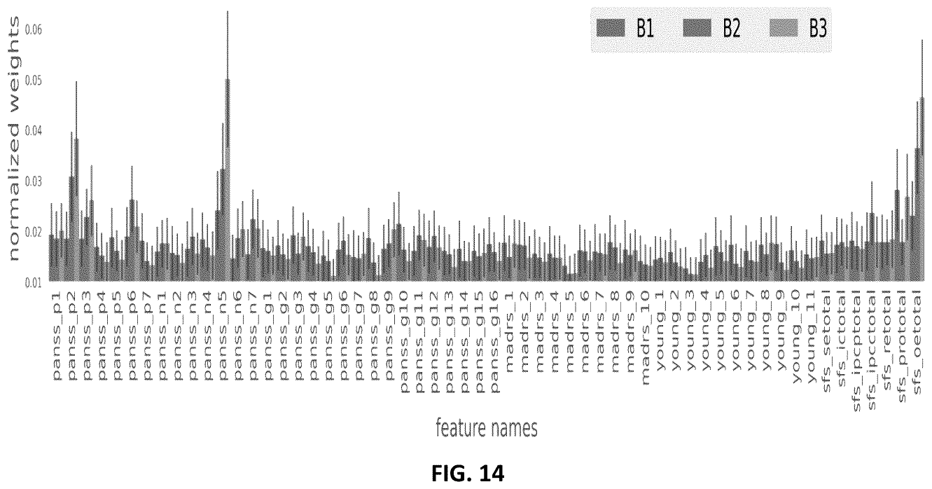

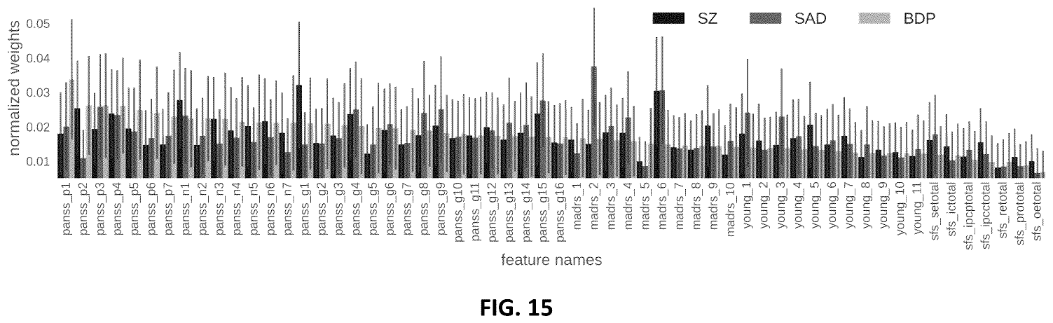

Next, the one-vs-all (BT1 or not, BT2 or not, BT3 or not) classification success was evaluated for extremely randomized ensembles of decision trees for each Biotype. The relative importance for Biotypes of every item for these component predictors (carrying out one-vs-all classification) is illustrated in FIG. 14. This same information for DSM is illustrated in FIG. 15. Inspection of these figures illustrates that no more than 20 items played substantial roles in differentiating groups. Therefore, in an aspect, to improve clinical efficiency, when constructing these decision trees, the maximum number of items used was restricted to 20. This implies that our model can consist of different combinations of tree-depth and number of trees, if the expected number of items encountered in each decision path is limited to an effective number of items (e.g., 20). Thus, for instance, there may be trees with depth 10 with 2 distinct estimators, or trees with depth 7 and 3 such estimators in our “forest”. It is important to note that for every model all 58 items had a chance to participate in the differentiation as a function of their success for classifying cases.

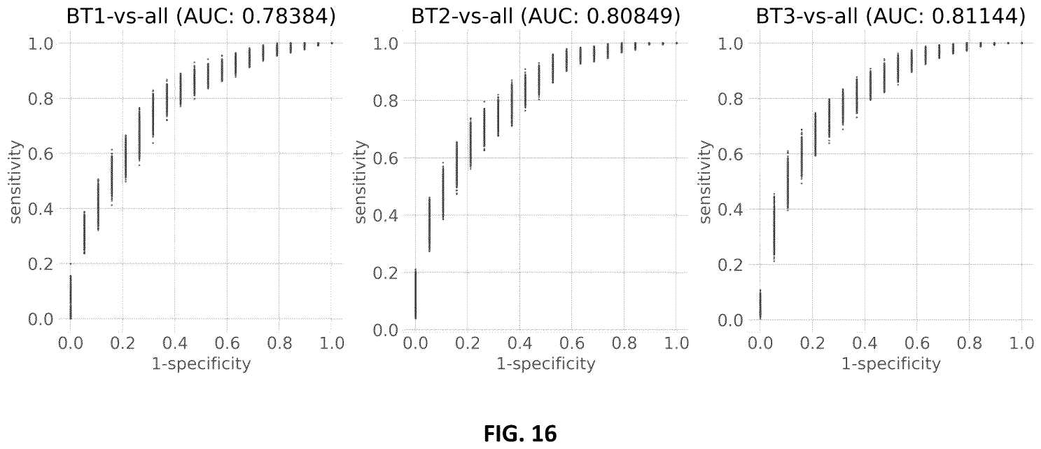

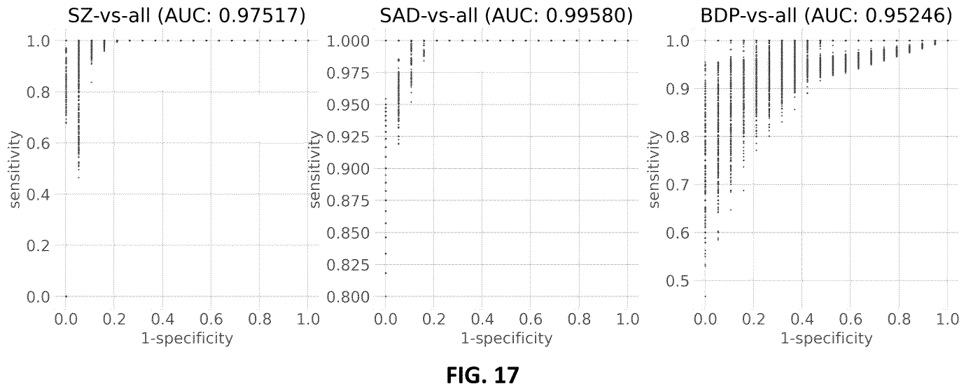

Optimizing which of the combinations yielded the best accuracy illustrates that trees with depth 7 or 8 and the number of estimators limited to 3 yield the best out-of-sample performance, when the maximum number of items presented is limited to 20. Furthermore, the mean number of items needed to obtain optimal classification accuracy is 10.6 (SD=0.93) for BT1, 10.5 (0.97) for BT2, and 10.5 (1.05) for BT3. The sensitivity, specificity, and AUC obtained for each Biotype in out-of-sample runs is illustrated in FIG. 16. This same information for DSM diagnoses is illustrated in FIG. 17. The classification of one Biotype versus all others has high sensitivity and specificity as illustrated in the ROC curves (and 99% confidence intervals), with corresponding AUCs also uniformly high (0.78 to 0.81). The classification of one DSM diagnosis versus all others has high sensitivity and specificity as illustrated in the ROC curves (and 99% confidence intervals), with corresponding AUCs also uniformly high (0.95 to 0.99).

### (iii) Pipeline for the Overall Classification Problem

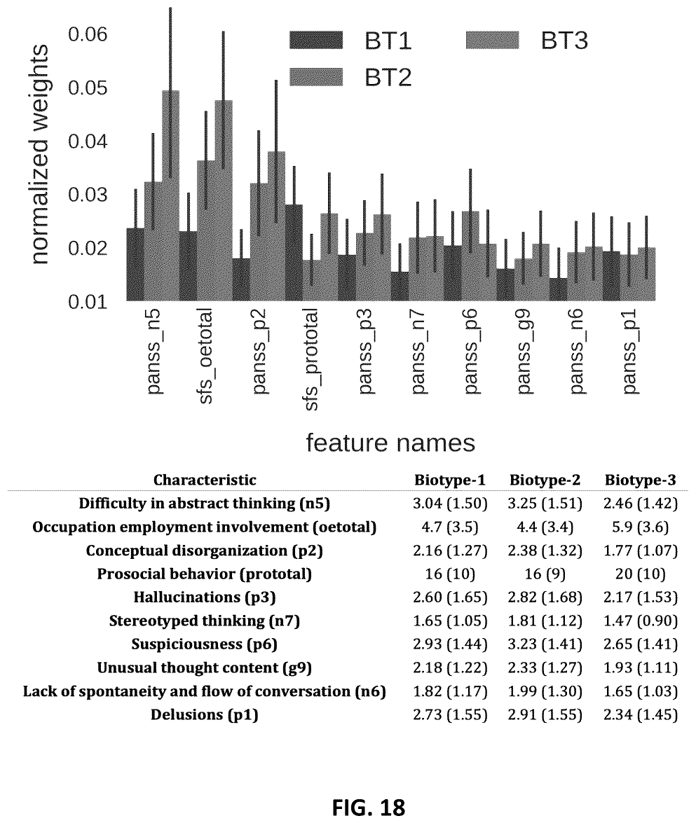

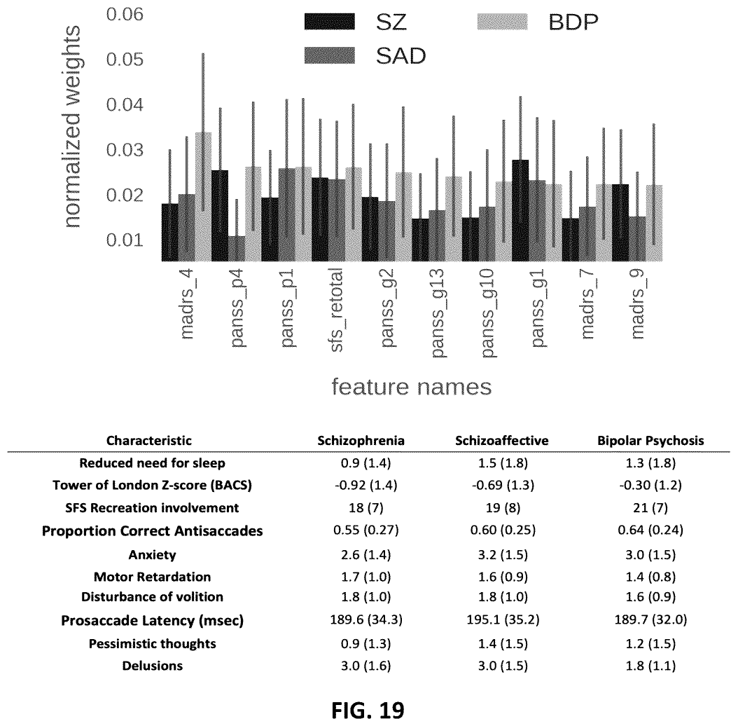

The inventors averaged over the feature importances of all models, which allowed for estimation of confidence bounds on the feature importances. The importance of the top 10 features in the end-to-end pipeline for B-SNIP Biotypes is illustrated in FIG. 18, along with the mean ratings (SDs) by Biotype of those individual features. The importance of the top 10 features in the end-to-end pipeline for DSM diagnoses is illustrated in FIG. 19, along with the mean ratings (SDs) by DSM diagnosis of those individual features. The top items, on average, for differentiating psychosis Biotypes were difficulty in abstract thinking, multiple indicators of social functioning (occupation employment involvement, prosocial behavior), conceptual disorganization, severity of hallucinations, stereotyped thinking, suspiciousness, unusual thought content, lack of spontaneous speech, and severity of delusions. Alternatively, the top items for differentiating DSM psychosis diagnoses were various indicators of physiological dysregulation (reduced need for sleep, excitement, anxiety, somatic complaints, lassitude), severity of delusions, level of recreational involvement, negative symptoms (volitional disturbance, blunted affect), disorientation, and pessimistic thoughts. Except for severity of delusions, there is no overlap in the top discriminating items for neurophysiologically anchored versus committee constructed diagnostic categories.

### Discussion of ADEPT-CLIN Outcomes

Fifty years ago, Carpenter, Strauss, and Bartko said “the failure of psychiatry to develop laboratory tests . . . limits the clarity and certainty [of] psychiatric classification.” Thirty years ago, Guze assumed psychiatry would transition to the inclusion of laboratory tests in differential diagnosis, like occurs in the rest of medicine. B-SNIP recently developed and replicated one possible laboratory testing approach, psychosis Biotypes, that accounts for previously contradictory findings within and across conventional clinical psychosis diagnoses.

It can be known if alternatives to conventional committee consensus diagnoses are better for etiological investigation and treatment targeting, but only if they are tested. This is the foundation of the scientific enterprise. In their published form, B-SNIP Biotype determinations require considerable time and specialized equipment for laboratory testing and biomarker quantification. However, for realistic translation to clinical settings, Biotype diagnosis must involve clinical assessment of signs and symptoms including a direct interview. This is always the first step in any medical evaluation. This invention illustrates that B-SNIP Biotypes map to constellations of clinical features, and that those mappings differ from the features differentiating conventional DSM diagnoses. Psychosis Biotypes have characteristic clinical features, so clinicians in any setting can estimate neurobiologically defined subgroups to facilitate comparative investigations of etiology and clinical utility.

To develop this innovative work, the inventors had 1907 cases with an idiopathic psychosis. Clinical ratings came from Birchwood, MADRS, PANSS, and YMRS rating scales. First, extremely randomized trees with 58 features were used to illustrate that psychosis Biotypes are differentiated via clinical evaluation. The overall accuracy of correctly assigning a case to their Biotype peaked at 91% accuracy with the administration of 57 items on average. This outcome illustrated and verified that neurobiologically defined psychosis subgroups have differentiating patterns of clinical characteristics.

Next, the inventors evaluated whether a reduced set of clinical features efficiently differentiates B-SNIP psychosis Biotypes. The sensitivities, specificities, and AUCs in out-of-sample runs for the more efficient clinical evaluations are uniformly high. The mean number of items for efficiently identifying a case in each subgroup versus all others (BT1 or not, BT2 or not, BT3 or not) was 10 to 11 for every comparison. This outcome illustrates an accurate B-SNIP Biotype determination is possible by using an efficient clinical evaluation.

On average, the top clinical characteristics for differentiating psychosis Biotypes, in order of importance, were difficulty in abstract thinking, multiple indicators of social functioning, conceptual disorganization, severity of hallucinations, stereotyped thinking, suspiciousness, unusual thought content, lack of spontaneous speech, and severity of delusions. Ratings of social involvement and clinical signs of cognition, thinking, and speech deviations are most important for differential Biotype categorization. BT1 and BT2 are remarkable for their more abnormal scores in relation to BT3 on every one of the differentiating features. BT2 are marginally the most deviant group across these clinical characteristics.

There are four important points regarding the list of clinical features, their differentiation of psychosis Biotypes, and their clinical relevance. First, these are not the items needed for determining whether a case has a psychosis; that question is answered using a different but related assessment. Second, these are the most important characteristics for differentiating between groups, on average. ADEPT is an adaptive algorithm, so the influence of items, and the order in which they are assessed, is unique to an individual case. Third, BT1 and BT2 were both different from BT3, with BT2 marginally the most clinically severe. Nevertheless, BT1 and BT2 cases apparently come by their clinical pictures via different physiologies. Attempts to optimally treat their clinical features may require different approaches despite the similarity in severity of clinical presentations. Fourth, DSM psychosis diagnoses are unable to capture these differentiating clinical characteristics or the different physiologies.

The clinical differentiators of psychosis Biotypes varied from those for DSM categories, so the two approaches are dissimilar in both neurobiology and presentation. In addition, the severity of reality distortions (hallucinations and delusions) was on average less important for distinguishing between Biotypes or DSM diagnoses. Dramatic and troubling clinical features like reality distortions should be targets of immediate intervention, like the symptomatic control of fever is always important regardless of its primary etiology. Similarly, the neurobiological correlates of reality distortions may transcend the physiological differences between psychosis subgroups. After control of the dramatic clinical manifestations, however, comes the search for underlying cause(s) of those signs and symptoms. ADEPT will allow clinical scientists in many settings to inform and identify the specific neurobiological features of psychosis Biotypes, and then use that information to select targeted and effective treatments.

The clinical portion of the ADEPT algorithm, via interaction with the B-SNIP psychosis Biotype database, yields Biotype membership for an individual case using widely available information. This allows clinicians and researchers to compare psychosis Biotypes to DSM categories for testing their differential efficacy in etiological and treatment investigations. The next iteration, ADEPT-COG, improves the probability of correct Biotype assignment using laboratory tests of behavioral and cognitive performance. This extension supports Guze's vision of transitioning psychiatry to a laboratory discipline.

### (B) the ADEPT-COGNITION (ADEPT-COG) Decision Tree Algorithm

The Bipolar-Schizophrenia Network for Intermediate Phenotypes (B-SNIP) original goals were to identify neurobiological features (i) shared across and (ii) distinctive to the major DSM psychosis diagnoses (schizophrenia, schizoaffective disorder, bipolar disorder with psychosis). The outcomes for DSM diagnoses across biomarker classes, however, only describe a neurobiological continuum (schizophrenia<schizoaffective disorder<bipolar disorder<community sample) with considerable group overlap and no evidence of neurobiological distinctiveness (FIG. 1).

As an alternative, B-SNIP searched for neurobiological similarity among persons with a psychosis of unknown origin, regardless of DSM diagnosis. Numerical taxonomy identified clusters of comparable individuals based on psychosis-relevant laboratory measures. B-SNIP identified, replicated, cross-validated, and externally validated transdiagnostic subgroups called psychosis Biotypes. Biotype-1 (BT1) and Biotype-2 (BT2) share marked cognitive deficits. Biotype-1's defining feature is weak neural response across multiple neurophysiological measures. Biotype-2's defining features are excessive intrinsic neural brain activity, like noisy static on an AM radio station, and the poorest general cognitive performance and motor inhibition. Biotype-3 (BT3) are closer to normal across cognitive and neurophysiological functions despite their psychosis, although have specific deviations on biomarkers indexing stimulus salience (FIG. 12). B-SNIP psychosis Biotypes do not fall along a simple continuum of neurobiological severity.

It is impractical for many clinical and research sites to collect and quantify the full B-SNIP biomarker panel. The inventors developed a procedure to efficiently assign an individual to their B-SNIP psychosis Biotype. This procedure, called Adaptive Diagnostics for the Efficient Prescription of Treatments (ADEPT), interacts with the processed B-SNIP biomarker database. The first iteration of ADEPT (ADEPT-CLIN, described above) illustrated that 10 to 11 adaptively selected clinical probes yield psychosis Biotype classifications with favorable sensitivities and specificities (≈0.80). B-SNIP psychosis Biotypes were formed using laboratory measures, but they also have characteristic clinical features. Clinical evaluation is the first step in psychiatric diagnosis, so ADEPT-CLIN is a low-burden B-SNIP psychosis Biotype estimator, even for under-resourced environments.

ADEPT-CLIN is an efficient Biotypes estimator, but there is room for increased precision (FIG. 13). Cognitive performance is an important differentiator of psychosis Biotypes. Cognitive dysfunction is often considered a core feature of schizophrenia, with evidence of cognitive difficulties predating symptom onset. But 25% to 30% of persons with schizophrenia or schizoaffective disorder have minimal to no cognitive deficits. Likewise, over 50% of bipolar psychosis cases have compromised cognitive performance. DSM psychosis diagnoses do not capture persons with this central correlate of illness severity and functional disability.

B-SNIP's cognition battery quantifies domains of importance to psychosis assessment: general cognition, goal maintenance, response speed, cognitive control, and inhibition. First, the Brief Assessment of Cognition in Schizophrenia (BACS) canvasses multiple cognitive domains (Verbal Memory, Processing Speed, Reasoning and Problem Solving, Working Memory) and provides an excellent measure of psychosis-related dysfunction. Second, antisaccade tests assess (i) inhibitory control, because the visual stimulus and required response location are incompatible (the correct response is to the mirror image location of a peripheral stimulus), and (ii) goal maintenance, because subjects must remember a response requirement over time. An initial glance toward an antisaccade stimulus is an error, which happens frequently in psychosis. Third, stop signal tests (SST) measure efficiency and adequacy of cognitive control when response preparation and the subsequent movement requirement are conflicted. Subjects see a ‘GO’ cue. On some trials, a ‘STOP’ signal is presented. Participants are instructed to GO (press a button) quickly unless they encounter a STOP signal, with inhibition a problem for some persons with psychosis.

Poor cognitive performance across multiple domains is peculiar to specific psychosis Biotypes, particularly BT1 and BT2 (FIG. 12). Measures of cognitive performance used by B-SNIP are also temporally stable in the absence of efforts to change them. These laboratory tests yield specific and useful targets for tracking cognitive changes in response to individually tailored interventions. Including cognitive functioning as additional features for psychosis Biotypes stratification appears promising for both diagnostic purposes and for tracking treatment success on domains of relevance to functional outcomes.

This is the second phase of this innovative work, an adaptive algorithm to diagnose psychosis Biotypes efficiently and accurately. This version of the innovative work is called ADEPT-COG and includes the 58 clinical features of ADEPT-CLIN plus scores from the BACS, saccade, and stop signal tests. The inventors illustrate that (i) cognitive test performance improves accuracy of B-SNIP psychosis Biotype diagnoses (improved sensitivity and specificity over clinical features alone); (ii) specific cognitive features optimize Biotype categorizations so tests can be preferentially selected for efficient diagnosis; and unexpectedly (iii) the same cognitive and clinical features do not differentiate Biotype and DSM diagnoses. Cognitive performance tests improve differentiation of B-SNIP Biotypes, so clinicians and researchers can use those features to both objectively diagnose and track treatment outcome for individual psychosis cases. Such information will support and amplify etiological investigations and treatment applications for persons with psychosis of unknown etiology.

### Method and Analyses

Recruitment details of the sample and its demographic characteristics are the same as reported above for ADEPT-CLIN.

### (i) Clinical Evaluations

Clinical assessment details and clinical characteristics of the sample are the same as those reported above for ADEPT-CLIN.

### (ii) Biotype Evaluations

Biomarker assessments and method of Biotype generation and assignment are the same as those reported above for ADEPT-CLIN.

(iii) Statistical Methods

The analytical approach from ADEPT-CLIN is extended in this paper to include cognition tests. The outcome of this innovative work is a low burden adaptive classifier of B-SNIP psychosis Biotypes using decision tree models. Clinical features are readily obtainable in any setting, and come from the seven subscales of the Birchwood, 10 items of the MADRS, 30 items of the PANSS, and 11 items of the YMRS, for a total of 58 clinical features. In addition to the clinical features, ADEPT-COG includes relatively low burden behavioral and cognition assessments. The added features are the six subscales and composite score of the BACS, three prosaccade reaction times (from gap, simultaneous, and overlap conditions), anti-saccade error and correct response latencies plus proportion of correct responses, and SST strategic slowing and proportion of SST errors. The Wide Range Achievement Test-IV reading subtest (WRAT), and participant age and sex are also included. This adds 18 measures to the 58 clinical features of ADEPT-CLIN.

One goal of the ADEPT innovative work is to reduce patient and clinician burden by reducing the number of features necessary for categorization while maintaining high classification accuracy. For the current analyses, an important distinction is the constraint on the number of features that may be used per sample. The model can be built from a relatively large set of items (the 76 total features available here). By using decision trees or ensembles of decision trees (a random forest), the number of estimators (trees) in each sample and the maximum depth (number of items, or features, assessed) of each tree can be constrained.

Constraints incur a performance cost. Ensembles and their related architectures can produce significant improvement over classical decision trees by effectively reducing the variance (in bagged ensembles or in random forests) or the bias (in boosted estimators) components of the test error. In the present innovative work, there is an ensemble with a limited number of estimators, each of which have a constrained depth. The result is a weighted combination of the component estimators, using weights optimized from the whole sample and the all 76-feature calibration data.

The “extra-trees” algorithm maximizes area-under-the-curve (AUC) for the constraint, outperforming other related classifiers. The extra-trees method drops the random forest idea of using bootstrap copies of the learning sample (thus reducing variance), and instead selects a cut-point at random, which leads to increased accuracy and decreased computational burden. To provide out-of-sample validation, the current method creates an empirical distribution for AUC, where each individual replicate is generated from a random split of the dataset into training and validation subsets. This approach is like “leave-p out cross-validation,” except a dataset fraction is specified instead of a fixed p (validation proportion=0.5, training proportion=0.5). The process is repeated until there is no significant change in the AUC distribution under a Kolmogorov-Smirnov test. Since this split can be modeled as a random draw from the complete dataset, confidence bounds for AUC are computed directly from percentiles of the empirical distribution.

In the present context, the three psychosis Biotypes (BT1, BT2, BT3) provide a multi-class identification problem. In the initial stage, the 76 features were used to develop a low-burden adaptive classifier that can be applied by non-B-SNIP sites to derive a Biotype classification. A one-vs-all extremely randomized ensemble of decision trees was then derived for each of the three Biotypes. While the out-of-sample AUCs obtained for each individual Biotype may be high, the end-to-end performance for the classifications is typically lower. In constructing these trees, a maximum allowable depth is enforced. For example, setting a maximum depth of 10, with 2 distinct estimators in our “forest”, implies that at most 20 features are used to decide the group membership of any given case. Depth means the number of choice points, e.g., if the individual says “yes” to a question, go to one set of queries, otherwise go to a different set of queries. This is an adaptive algorithm, so administered items are determined based on the next most useful bit of information given the response to previous information. The average number of features used for the overall classification of a case into one of the three Biotypes typically will be smaller than the maximum allowable depth. These same procedures were also repeated for the problem of determining a DSM diagnosis (schizophrenia, schizoaffective disorder, bipolar I disorder with psychosis). The outcome of this procedure, referred to herein as ADEPT-COG, is described below.

### Results

### (i) Overall Accuracy of ADEPT-COG as a Function of Items

The first step is determining the model's overall classification accuracy as a function of the number of items included in the Biotypes classification problem. FIG. 13 illustrates mean accuracy (with 99% confidence intervals) for classifying a case into one of the three Biotypes as a function of items used (the 76 total features). Overall accuracy of correctly assigning an individual case to their Biotype membership peaks at 94.6% correct with 65 items presented on average. There is a local maximum in mean out-of-sample classification accuracy of 90.5% with 18 items presented, which shows a more limited evaluation can achieve successful Biotype classification. The overall Biotypes classification accuracy is significantly enhanced by the inclusion of behavioral and cognitive features over the use of clinical features alone (see FIG. 13). FIG. 13 shows this same information for DSM psychosis diagnoses. There is no increase in DSM diagnostic accuracy by adding behavioral and cognitive features to the optimal clinical assessment.

### (ii) Accuracy of ADEPT-COG Individual Biotype Assignment

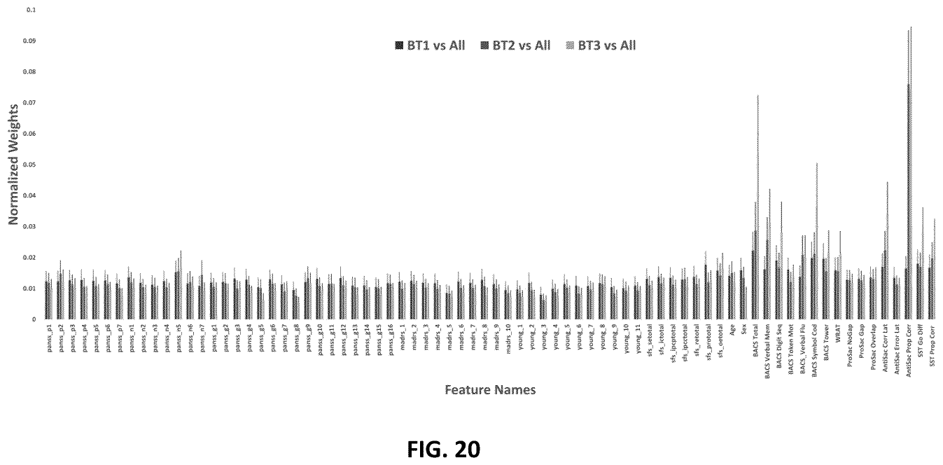

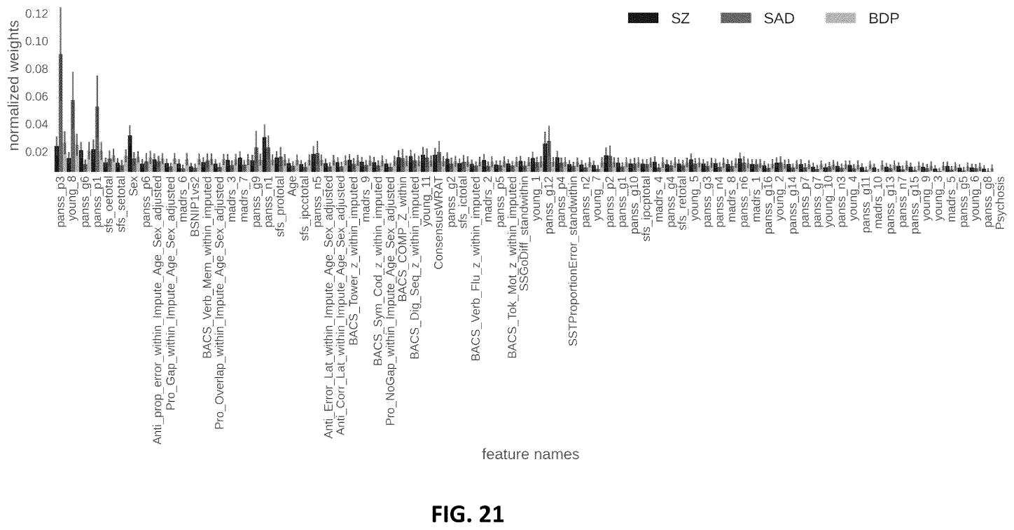

Next, the inventors determined the one-vs-all (BT1 or not, BT2 or not, BT3 or not) classification success for extremely randomized ensembles of decision trees for each Biotype. The relative importance for Biotypes diagnosis of every item (carrying out one-vs-all classification) is shown in FIG. 20. The relative importance for DSM diagnosis of every item (carrying out one-vs-all classification) is shown in FIG. 21. The inspection of this figure indicates that no more than 20 items played substantial roles in differentiating Biotypes. Therefore, to improve efficiency, when constructing these decision trees, the maximum number of items is restricted to 20. This implies that the model can consist of different combinations of tree-depth and number of trees, if the expected number of items encountered in each decision path is limited to 20. Thus, for instance, there can be trees with depth 9 with 2 distinct estimators, or trees with depth 6 and 3 such estimators in our “forest”. It is important to note that for every model all 76 features had a chance to participate in the differentiation as a function of their success for classifying cases.

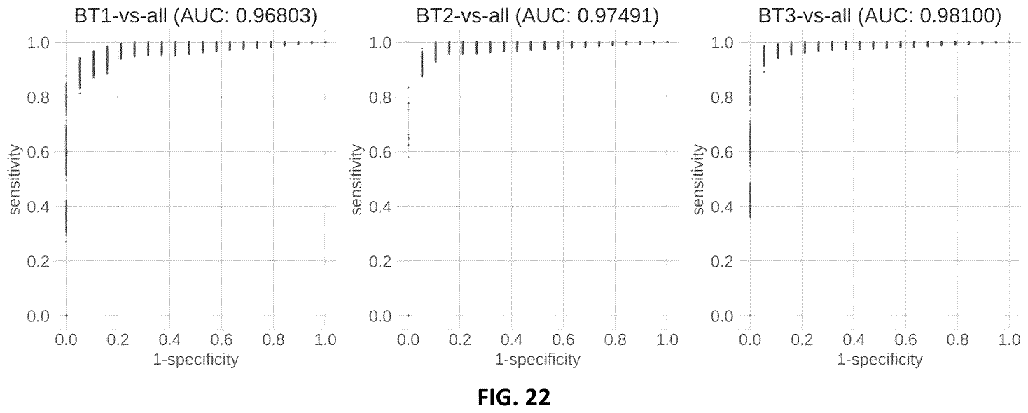

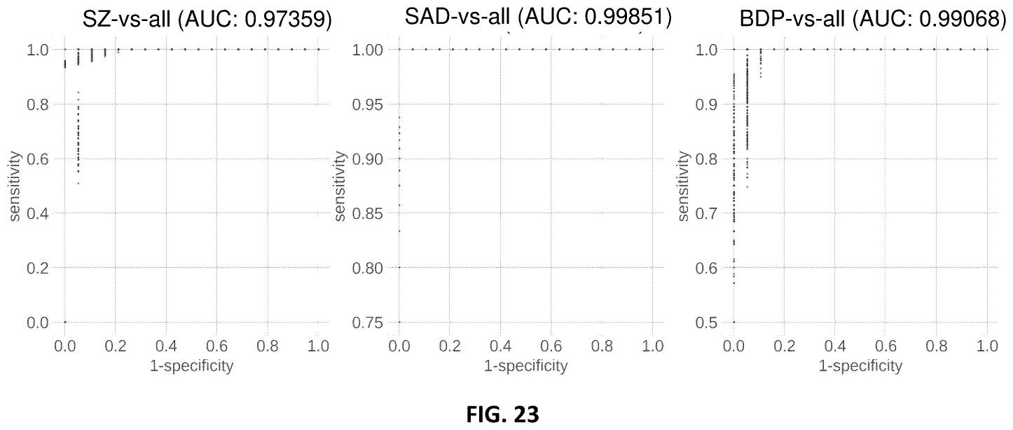

Investigating which of the combinations yielded the best accuracy illustrates that trees with depth 6 or 7 and the number of estimators limited to 3 yield the best out-of-sample performance, when the maximum number of items presented is limited to 20. The mean number of items observed to obtain optimal classification accuracy was 9.71 (SD=2.60) for BT1, 8.91 (2.63) for BT2, and 8.63 (2.61) for BT3. The sensitivity, specificity, and AUC obtained for each Biotype in out-of-sample runs is illustrated in FIG. 22. The sensitivity, specificity, and AUC obtained for each DSM diagnosis in out-of-sample runs is illustrated in FIG. 23. The classification of one Biotype versus all others has high sensitivity and specificity as illustrated in the ROC curves (and 99% confidence intervals), with corresponding AUCs also uniformly high (0.96 to 0.98). These AUC values are higher than those obtained when using clinical features in ADEPT-CLIN.

### (iii) Pipeline for the Overall Classification Problem

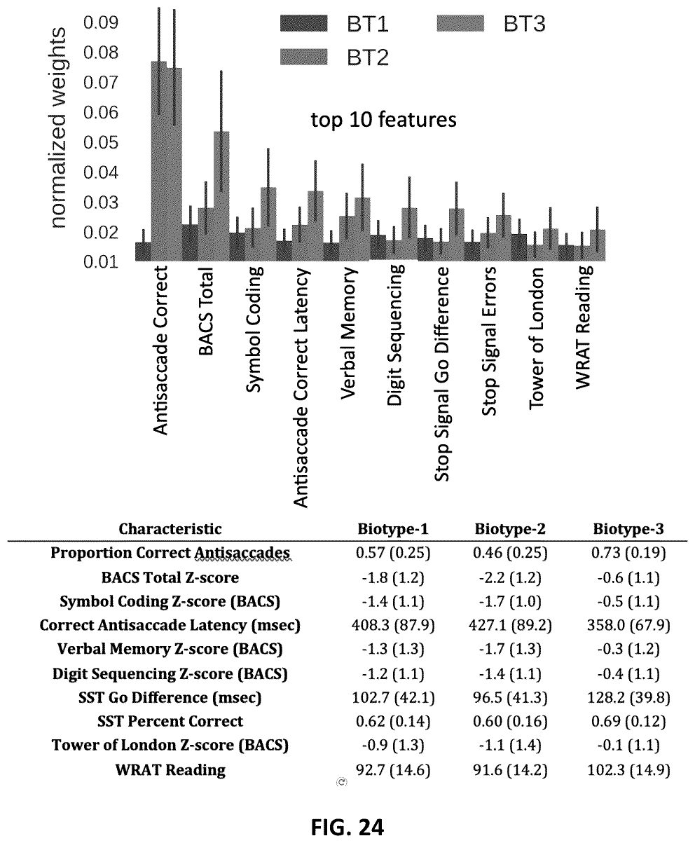

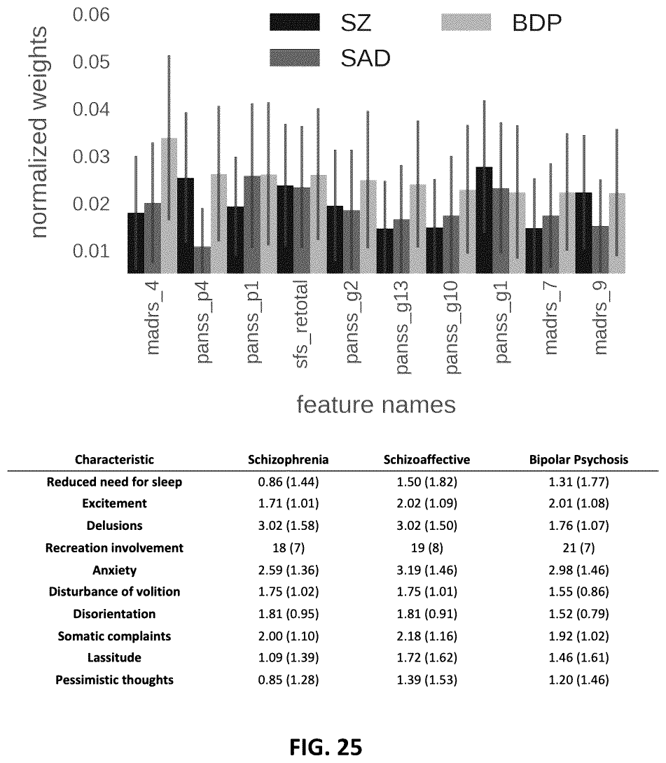

The inventors averaged over the feature importances of all models, which allowed us to estimate confidence bounds on the feature importances. The importance of the top 10 features in the end-to-end pipeline is illustrated in FIG. 24, along with the mean ratings (SDs) by Biotype of those individual features. This same information for DSM diagnoses is illustrated in FIG. 25. The top items, on average, for differentiating B-SNIP psychosis Biotypes were correct antisaccade responses, total BACS score, symbol coding, latency of correct antisaccades, verbal memory, digit sequencing, SST go reaction time difference, SST proportion correct responses, Tower of London, and the WRAT. Alternatively, the top items for differentiating DSM psychosis diagnoses were reduced need for sleep (MADRS 4), Tower of London, SFS recreational engagement, correct antisaccade responses, anxiety (PANSS G2), motor retardation (PANSS G7), volitional disturbance (PANSS G13), prosaccade no gap reaction time, pessimistic thoughts (MADRS 9), and delusions (PANSS P1). Except for correct antisaccade responses and Tower of London, there is no overlap in the top discriminating items for neurophysiologically anchored versus committee constructed diagnostic categories. The top discriminators for psychosis Biotypes are all laboratory measures, while seven of the top 10 DSM discriminators are clinical features. Both outcomes are consistent with the methods underlying the construction of the classification schemes.

### Discussion of ADEPT-COG Outcomes

Psychosis volunteers were recruited across B-SNIP sites to generate a research sample with comprehensive clinical evaluations and extensive biomarker data. Participants received conventional psychosis diagnoses based on clinical evaluations, as well as Biotype designations based on B-SNIP bio-factor scores. The sample is unique and captures a broad biomarker characterization across individuals with an unexplained psychosis. The sample's bio-factor and Biotype outcomes independently replicated, cross-validated, and show favorable construct validity in relation to DSM diagnoses. Importantly, biomarker features are generated agnostic to clinical characterization and B-SNIP Biotypes are largely independent of DSM diagnosis, other than presence of an unexplained psychosis.

The burden of B-SNIP's comprehensive strategy is its time-, information-, and resource-intensiveness. The practical and technical requirements of collecting the comprehensive biomarker battery and subsequent scoring and analyses is too involved for most settings, making it unrealistic to broadly implement this system. B-SNIP psychosis Biotypes are promising for facilitating and identifying practical, functionally specific, and valid treatment targets not derivable from any other approach. The only way to evaluate whether laboratory-based psychosis diagnosis can improve treatment targeting and patient outcomes is to test this prospect, which is the basis of the clinical scientific enterprise.

The ADEPT innovative work minimizes diagnostic burden and allows any clinical research team to evaluate the utility of psychosis Biotypes as etiological probes and treatment targets. This innovative work can be implemented for large scale projects and less-resourced environments. ADEPT is a “reverse translation” process, starting with the known group (e.g., a psychosis Biotype), that specifies the most impactful and efficient features for psychosis categorization. ADEPT outcomes can yield both psychosis Biotype and DSM diagnoses. This classifier provides a widely implementable system to meet the challenges of testing the utility of “bio-diagnoses” within psychiatry.

The first iteration, ADEPT-CLIN, uses common clinical ratings and extremely randomized trees to show that neurobiologically defined psychosis subgroups have differentiating patterns of clinical characteristics. Biotype differentiation using clinical features alone is efficient (only 10-11 items are preferably utilized) and accurate for a minimal time and technological investment (AUCs under the sensitivity-specificity curve of ≈0.80). The clinical (and cognitive) features important for Biotype and DSM diagnoses are surprisingly and remarkably different, illustrating that these systems are not overlapping for understanding etiology and selecting optimal treatments.

The next step in ADEPT development is to add cognitive features to the clinical evaluation. Psychosis-relevant measures of cognition are important and multifaceted differentiators of some psychosis subgroups. Many cognition-relevant tests are either already available in most clinical research settings or can be implemented with modest setting, staff, and patient burden. As illustrated in this innovative work, ADEPT-COG shows remarkable accuracy for a one versus all psychosis Biotype diagnosis (e.g., BT1 versus others), with uniformly high AUCs of greater than 0.95. This is a substantial enhancement over ADEPT-CLIN. This increased accuracy was achieved using an efficient number of items (8-9 on average) which minimizes overall burden.

In comparison, adding cognitive features to the most efficient clinical evaluation unexpectedly did not enhance DSM diagnostic accuracy. In the development of DSM-5, the Psychosis Working Group stated they excluded cognition for differential psychosis diagnosis because of specificity concerns. On the one hand, this ADEPT-COG outcome supports that prescient decision. The top psychosis Biotype discriminators are laboratory measures, but seven of the top 10 DSM discriminators are clinical ratings. This difference is consistent with how the two classification systems were constructed, with relatively little contribution of clinical features for Biotypes over cognition and vice versa for DSM diagnosis. On the other hand, and contrary to expectation, the most efficient clinical evaluation for differential diagnosis of schizophrenia, schizoaffective disorder, and bipolar disorder deviates from DSM criteria.

### General Discussion of ADEPT Outcomes

There are a few important considerations associated with implementing the ADEPT innovative work. First, ADEPT identifies the most important features for group differentiation, not for identifying the presence of a clinically significant psychosis. That goal is efficiently achieved in other ways. Second, the top 10 items are an average of feature importances over diagnostic decisions. ADEPT is adaptive, so the most important items and their administration order is unique to an individual case. Third, ADEPT does not substitute for carefully evaluating patients on multiple dimensions of relevance to their clinical care. For instance, troubling reality distortions should be targets of immediate intervention, although such psychosis symptoms may have different causes across individuals.

Psychiatry has anticipated the value of biologically based diagnoses, like other disciplines where biology explains disease. Instead of only diagnosing dropsy, pyrexia or epilepsy, there are known specific conditions and etiological mechanisms that require different treatment despite superficial similarities of clinical characteristics. Biological diagnoses may facilitate a deeper, more nuanced understanding of disease, including serious psychiatric conditions. It is reasonable to assume that known neural pathologies will promote and facilitate novel and effective treatment developments. The ADEPT innovative work facilitates those goals.

Specific neurobiological targets, like those identified via B-SNIP psychosis Biotypes, may be useful for constructing focused treatment trials, teaching clinical care, and generating novel disease understanding. For instance, ADEPT may make a Biotypes strategy practically useful for large pharmacological trials to compare drug actions as a function of different diagnostic schemes (Biotypes versus DSM diagnoses). ADEPT also allows for the efficient implementation of extensive biomarker knowledge in etiological investigations (e.g., large scale genomics or imaging consortia). Because ADEPT provides a direct link to such knowledge, this innovative work find application in in new drug testing, championed by pharmaceutical companies. This innovative work, therefore, provides a means of creating biologically relevant subject groups and testing for pharmacological specificity with new agents.

### III. Computer Hardware Components

The present systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed, and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, can run many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is like multi-tasking computing, as the terms task and process are often used interchangeably, although some operating systems make a distinction between the two.

The present technology may be a system, a method, and/or a computer program product at any possible technical detail level of integration. The computer program product may include a computer readable storage medium (or media) having computer readable program instructions thereon for causing a processor to carry out aspects of the present innovative work. The computer readable storage medium can be a tangible device that can retain and store instructions for use by an instruction execution device. In some aspects, the computer readable storage medium can be a tangible device that can retain and store instructions for use by an instruction execution device to perform any of the operations discussed herein regarding FIGS. 1-27.

Preferably, in all instances described in this technology, the computer-readable storage medium or computer-readable medium is a non-transitory computer-readable storage medium or a non-transitory computer-readable medium, respectively.

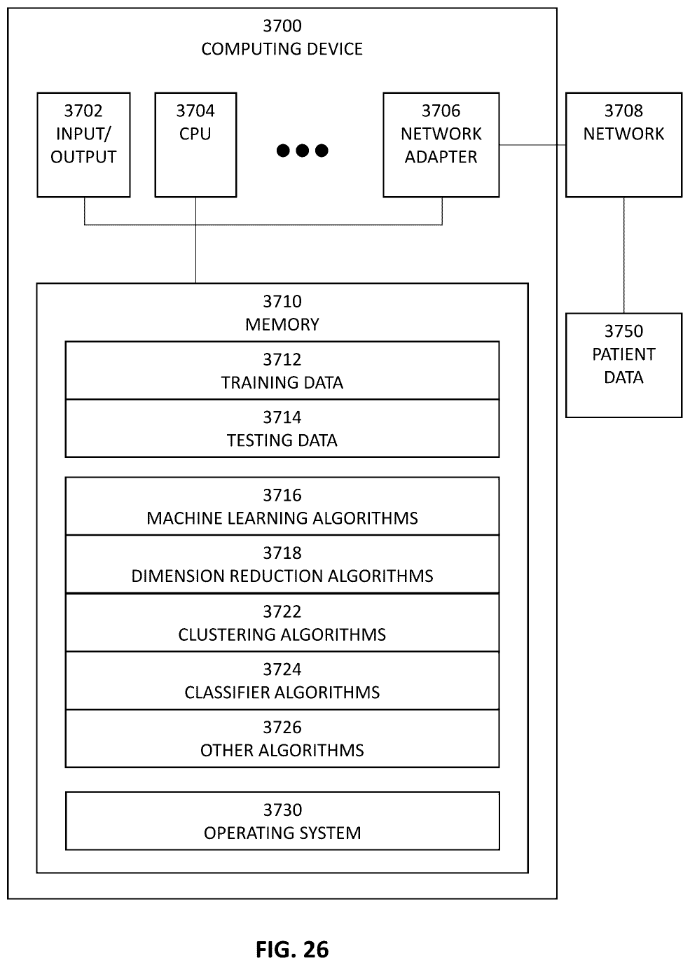

FIG. 26 illustrates a computing device 3700 as it relates to the present disclosure. The computing device 3700 may, for example, perform calculations, execute routines and algorithms, process data, communicate with other devices via a network, and display results. For example, a computing device 3700 may comprise a processor or CPU 3704, a network adapter 3706 for communication with a network 3708. The network 3708 may connect the computing device 3700 to external data sources such as patient data 3750 or to other computers (not shown in the figure). The computing device may comprise an input/output device 3702. Such an input/output component 3702 may be an input device, an output device, or both and the computing device 3700 may have several such components. Example input devices 3702 include a keyboard, a mouse, a microphone, a touchpad, a joystick, and the like. Example output devices 3702 include a display, a speaker, a haptic feedback device, and the like. The computing device 3700 may further comprise memory 3710 or a computer readable storage medium 3710. In the computer memory 3710 may reside instructions for carrying out the methods and techniques described elsewhere in this disclosure. The computer memory 3710 may also comprise an operating system 3730 for control of the various parts and components of the computing device 3700. The memory 3710 may also store data, for example training data 3712 and testing data 3714. The memory 3710 may also comprise algorithms such as machine learning algorithms 3716, dimension reduction algorithms 3718, decision tree algorithms 3720, clustering algorithms (e.g., k-means), 3722, classifier algorithms 3724, or other algorithms 3726.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present innovative work may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry to perform aspects of the present innovative work.

Aspects of the present innovative work are described herein with reference to flowchart illustrations and/or block diagrams of methods, apparatus (systems), and computer program products according to embodiments of the innovative work. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks.

These computer readable program instructions may also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart or block diagram block or blocks.

The flowchart and block diagrams in FIG. 26 illustrate the architecture, functionality, and operation of possible implementations of systems, methods, and computer program products according to various embodiments of the present innovative work. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in FIG. 26. For example, two blocks shown in succession may, in fact, be executed substantially concurrently, or in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts, or that carry out combinations of special purpose hardware and computer instructions.

Although specific embodiments of the present innovative work have been described, it will be understood by those of skill in the art that there are other embodiments that are equivalent to the described embodiments. Accordingly, it is to be understood that the innovative work is not to be limited by the specific illustrated embodiments, but only by the scope of the appended claims.

### Non-Limiting Flow Chart for Implementation of the Innovative Work

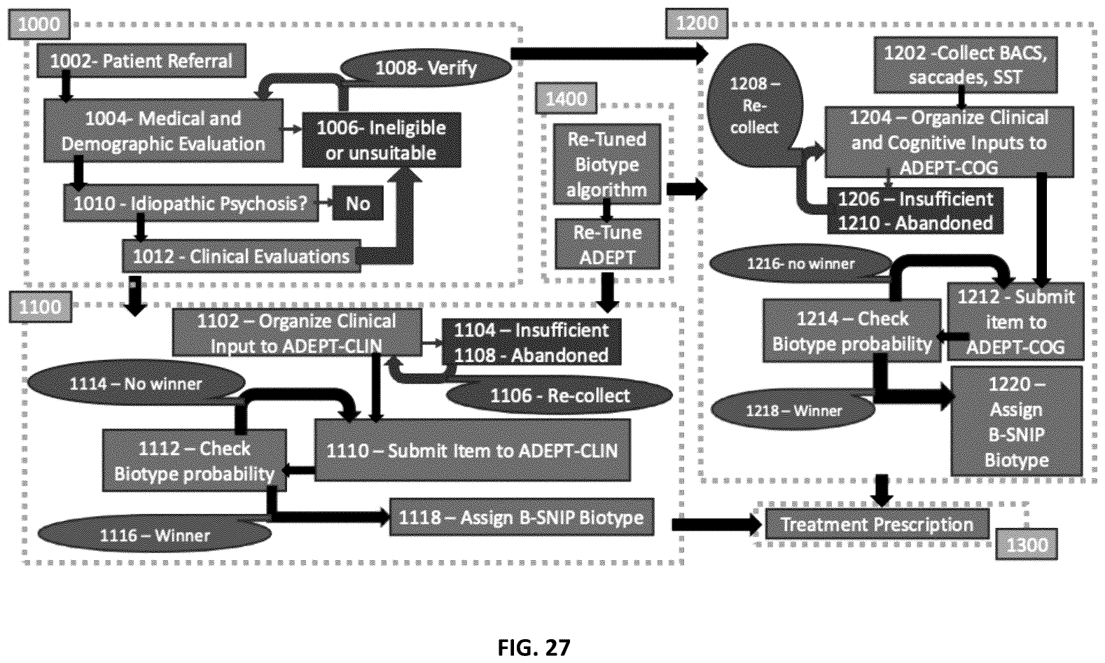

FIG. 27 shows a method involved in implementing the innovative work as described above. Some of the steps, specifically those outlined inside the dotted 1000 box, occur prior to implementing the innovative work. In some aspects, these steps are important prerequisites. First, it must be determined whether a patient is suitable for the innovative work (box 1000). A patient is first referred for assessment (step 1002). The patient's medical and demographic information is collected and reviewed (step 1004), as is done in evaluations conducted prior to any medical diagnosis. From here, the patient must be verified to be suitable for further evaluation. There are many ways a patient is deemed unsuitable for a B-SNIP psychosis Biotype diagnosis, including age (less than 18 years of age or greater than 65 years of age), having suffered serious head injury, being a chronic or active user of psychoactive substances that compromise the accuracy of clinical evaluations and laboratory tests of brain function, or having other conditions that better account for the clinical picture, for instance a neurological disorder such as temporal lobe epilepsy, known genetic deviation (e.g., 22q11 deletion syndrome) or autoimmune condition (e.g., anti-NMDA receptor autoimmune encephalopathy). Upon finding that a patient is deemed unsuitable (step 1006), this finding is verified prior to definitive exclusion from B-SNIP psychosis Biotype evaluation (step 1008).

When a referred patient successfully passes the above screening, the patient is then evaluated for psychosis (step 1010), which is defined as perceptions, thoughts, or actions that do not comport with socially shared experience. Psychosis can be verified using a standard evaluation that identifies the presence of delusions and hallucinations (collectively called reality distortions), thoughts in which speech is difficult or impossible to comprehend (e.g., thought disorder), or unusual behaviors that do not fit the situation (e.g., so-called negative symptoms or catatonia). The patient must meet the clinical psychosis standard to be evaluated for a psychosis Biotype; otherwise, they are excluded from B-SNIP psychosis Biotype evaluation (red box “No”). If the patient meets the psychosis definition, clinical evaluations important to implementing the innovative work are conducted (step 1012).

After successful completion of box 1000, ADEPT-CLIN (box 1100) can be implemented. First, the clinical information can be organized into a format preferred for implementation of ADEPT-CLIN (step 1102). Ratings of good quality are prepared according to techniques developed for each individual clinical scale (step 1102). If the clinical information is insufficient because of missing items or out-of-scale ratings (step 1104), then the clinical information is re-collected (step 1106). If the data are still insufficient (e.g., the patient is not available, will not answer clinical probes, or is unable to respond sufficiently to rate an item), then the evaluation is abandoned (step 1108).

After verifying the clinical information is acceptable and is organized into the proper format, this information is submitted to ADEPT-CLIN using the highest probability item for group discrimination (step 1110). The probability of the patient belonging to one psychosis Biotype versus all others is then checked (step 1112). This check is done in relation to the maximal probabilities obtained for optimal AUC for the one-versus-all decision as described above. The patient's score on the input to ADEPT-CLIN is compared to patients in the processed B-SNIP psychosis Biotype database to determine the match between the patient's score and scores of patients in this database with a known psychosis Biotype. If there is no clear Biotype diagnosis winner for a Biotype determination (step 1114), then the next most useful item along with previous items is submitted to ADEPT-CLIN (step 1110). The probability of the patient belonging to one psychosis Biotype versus all others is re-checked (step 1112). This procedure is repeated until there is a clear winner for a Biotype determination in the one-versus-all decision (step 1116). When there is a clear winner (there is no further change in the Biotype diagnosis probability), the Biotype is assigned to the patient (step 1118) and the treatment(s) relevant to the Biotype are implemented (box 1300).

After successful completion of box 1000, ADEPT-COG (box 1200) can be implemented. First, the patient completes the BACS, pro- and anti-saccades, and SST (step 1202) according to methods described above, in multiple previous publications, and in International Patent Application No. PCT/US2025/020864, the contents of which are herein incorporated by reference in their entirety. Details of BACS, saccade, and SST quantification are also detailed in those locations. The clinical and cognitive information can then be organized into a format preferred for implementation of ADEPT-COG (step 1204). Data of good quality are then processed according to techniques developed for each individual clinical rating or cognitive measure (step 1204). Those processing techniques include creating scores for every clinical rating and grand averages for every subcomponent of the cognitive measures, using the approaches outlined above and in previous publications.

If the clinical or cognitive information is insufficient because of missing items or out-of-scale ratings (step 1206), then the insufficient information is re-collected (step 1208). If the relevant data are still insufficient (e.g., the patient is not available, will not answer clinical probes, cannot complete the cognitive testing, or is unable to respond sufficiently to rate an item), then the evaluation is abandoned (step 1210). After verifying the clinical and cognitive information is acceptable and is organized into the proper format, this information is submitted to ADEPT-COG using the highest probability item for group discrimination (step 1212). The probability of the patient belonging to one psychosis Biotype versus all others is then checked (step 1214). This check is done in relation to the maximal probabilities obtained for optimal AUC for the one-versus-all decision as described above. The patient's score on the input to ADEPT-COG is compared to patients in the processed B-SNIP psychosis Biotype database to determine the match between the patient's score and scores of patients in this database with a known psychosis Biotype. If there is no clear winner (a psychosis Biotype diagnosis versus all others) for a Biotype determination (step 1216), then the next most useful item along with previous items is submitted to ADEPT-COG (step 1212). The probability of the patient belonging to one psychosis Biotype versus all others is re-checked (step 1214). The procedure is repeated until there is a clear winner for a Biotype determination in the one-versus-all decision (step 1218). When there is a clear winner, the Biotype is assigned to the patient (step 1220) and the treatment(s) relevant to the Biotype are implemented (box 1300).

The outputs of ADEPT are probabilities that the patient has a B-SNIP psychosis Biotype versus all other Biotypes (e.g., BT1 or not) given the items submitted to ADEPT. An individual patient's psychosis B-SNIP Biotype diagnosis is the Biotype with the highest probability when that patient's ADEPT input is compared to patients in the B-SNIP psychosis Biotype database who have a known Biotype based on complete biomarker data. In this way, it is possible using ADEPT to obtain a B-SNIP psychosis Biotype even in the absence of complete data.

ADEPT can be modified with the addition of a sufficiently large number of new patients or the addition of new clinical or biomarker measures (e.g., like the structural MRI algorithm for BT1 as described above and the signal-to-noise ratio measure as described above). In this aspect, processed data with new subjects or new biomarkers are used to re-tune the psychosis Biotypes algorithm (box 1400) in International Patent Application No. PCT/US2025/020864, the contents of which are herein incorporated by reference in their entirety.

Upon re-tuning of the B-SNIP psychosis Biotypes algorithm, ADEPT can be re-tuned to meet this refined diagnostic standard (box 1400). New and previous clinical and cognitive features as defined by the re-tuned Biotypes algorithm are used to re-tune ADEPT. This is done by determining in the processed B-SNIP psychosis Biotypes databased which items maximize the probability of a Biotypes diagnosis in relation to all others as described above. This yields updated ADEPT item ordering of step 1102 and step 1204 and changes the probability distributions for determining a Biotype diagnosis, thus modifying the ADEPT-CLIN computations of steps 1110, 1112, 1114, and 1116, and the ADEPT-COG computations of steps 1212, 1214, 1216, and 1218. The re-tuned ADEPT algorithm modifies the Biotype probabilities.

The outcome of ADEPT and the generation of a B-SNIP psychosis Biotype diagnosis (step 1118 and step 1220) is that the patient can be prescribed a specific treatment or treatments that are appropriate for that diagnosis to rectify specific issues (box 1300). Examples of such treatments are provided above. Some of those treatments may be applied by the inventors (e.g., the sensory training intervention for BT1), but most frequently they will be implemented by the referring clinician.

The above-described methods and/or process(es) shown in figures presented in this disclosure may be implemented via any of the computing devices, systems, or components described herein.

The disclosed systems and methods of use can be further understood through the following enumerated paragraphs or embodiments.
1. A computer-implemented method (CIM) for neurobiological diagnosis of a subject, optionally followed by an individual devising a treatment regiment, the method comprising:
   using one or more adaptive diagnostic algorithms configured to (i) process the subject's clinical information, cognitive information, and/or behavioral information to obtain a first score and (ii) compute and/or output a probability of the subject having a subtype of psychosis by matching the first score with corresponding scores of other subjects with a known subtype of psychosis, wherein the one or more adaptive diagnostic algorithms have been trained on a second set of clinical information, cognitive information, and/or behavioral information to recognize one or more subtypes of psychosis, optionally wherein training of the one or more adaptive diagnostic algorithms occurs on a computing device, and
   wherein the one or more adaptive diagnostic algorithms are operably linked to one or more processors capable of executing the one or more adaptive diagnostic algorithms.
2. The CIM of paragraph 1, wherein the one or more adaptive diagnostic algorithms are operably linked to (i) a device configured to present audio data, visual data, audio-visual data, optical data, electrical data, magnetic data, electromagnetic data, mechanical data or a combination thereof, related to neurobiological diagnosis.
3. The CIM of paragraph 1 or 2, wherein the one or more adaptive diagnostic algorithms are configured to process the subject's clinical information.
4. The CIM of any one of paragraphs 1 to 3, wherein the one or more adaptive diagnostic algorithms are configured to process the subject's clinical information and cognitive information.
5. The CIM of any one of paragraphs 1 to 4, wherein the one or more adaptive diagnostic algorithms are configured to process the subject's clinical information, cognitive information, and behavioral information.
6. The CIM of any one of paragraphs 1 to 5, wherein the one or more adaptive diagnostic algorithms include a randomized ensemble of classifiers.
7. The CIM of any one of paragraphs 1 to 6, wherein the one or more adaptive diagnostic algorithms include an adaptive decision tree algorithm.
8. The CIM of any one of paragraphs 1 to 7, wherein the one or more adaptive diagnostic algorithms include an extra-trees classifier.
9. The CIM of any one of paragraphs 1 to 8, step (ii) is performed iteratively until a specific subtype of psychosis receives a highest probability.
10. The CIM of any one of paragraphs 1 to 9, wherein using the subject's cognitive information to compute the probability is performed after using the subject's clinical information.
11. The CIM of any one of paragraphs 1 to 10, wherein the psychosis is idiopathic.
12. The CIM of any one of paragraphs 1 to 11, wherein the subtype of psychosis includes a neural dysregulation Biotype (BT2), a neural vigor Biotype (BT1), and/or a stimulus salience Biotype (BT3).
13. The CIM of any one of paragraphs 1 to 11, further involving:
   causing a recommendation of one or more treatment regiments based on the subtype of psychosis to be presented at a user interface comprising a display of a computing device, an electro-mechanical acoustic system, or a combination thereof.
14. The CIM of any one of paragraphs 1 to 13, wherein the one or more adaptive diagnostic algorithms are capable of being re-tuned using a third set of clinical information, cognitive information, behavioral information, bio-factor data, or a combination thereof.
15. The CIM of any one of paragraphs 1 to 14, wherein the CIM provides or is capable of providing neurobiological diagnosis in real-time.
16. The CIM of any one of paragraphs 1 to 15, involving an individual devising a treatment regimen, wherein the treatment regimen includes antipsychotic medications (typical and atypical), mood stabilizers, anxiolytics, antidepressants; cognitive behavioral therapy for psychosis; supportive therapy; insight-oriented therapy; family therapy; social skills training; vocational rehabilitation; case management; hospitalization; electroconvulsive therapy (ECT); sleep hygiene; exercise; dietary adjustments; mindfulness; relaxation techniques; or any combinations thereof.
17. A non-transitory computer-readable medium (CRM) with one or more computer-executable instructions stored thereon executed by one or more processors, wherein the one or more computer-executable instructions include one or more adaptive diagnostic algorithms configured to (i) process a subject's clinical information, cognitive information, and/or behavioral information to obtain a first score and (ii) compute and/or output a probability of the subject having a subtype of psychosis by matching the first score with corresponding scores of other subjects with a known subtype of psychosis, wherein the one or more adaptive diagnostic algorithms have been trained on a second set of clinical information, cognitive information, and/or behavioral information to recognize one or more subtypes of psychosis, optionally wherein training of the one or more adaptive diagnostic algorithms occurs on a computing device, and
   wherein the one or more computer-executable instructions are operably linked to the one or more processors.
18. The non-transitory CRM of paragraph 17, wherein the one or more computer-executable instructions are operably linked to a device configured to present audio data, visual data, audio-visual data, optical data, electrical data, magnetic data, electromagnetic data, mechanical data, or a combination thereof, related to neurobiological diagnosis.
19. The non-transitory CRM of paragraph 17 or 18, wherein the one or more adaptive diagnostic algorithms are capable of being re-tuned using a third set of clinical information, cognitive information, behavioral information, bio-factor data, or a combination thereof.
20. The non-transitory CRM of any one of paragraphs 17 to 19, wherein the one or more adaptive diagnostic algorithms are configured to process the subject's clinical information.
21. The non-transitory CRM of any one of paragraphs 17 to 20, wherein the one or more adaptive diagnostic algorithms are configured to process the subject's clinical information and cognitive information.
22. The non-transitory CRM of any one of paragraphs 17 to 21, wherein the one or more adaptive diagnostic algorithms are configured to process the subject's clinical information, cognitive information, and behavioral information.
23. The non-transitory CRM of any one of paragraphs 17 to 22, wherein the one or more adaptive diagnostic algorithms include a randomized ensemble of classifiers.
24. The non-transitory CRM of any one of paragraphs 17 to 23, wherein the one or more adaptive diagnostic algorithms include an adaptive decision tree algorithm.
25. The non-transitory CRM of any one of paragraphs 17 to 24, wherein the one or more adaptive diagnostic algorithms include an extra-trees classifier.
26. The non-transitory CRM of any one of paragraphs 17 to 25, step (ii) is performed iteratively until a specific subtype of psychosis receives a highest probability.
27. The non-transitory CRM of any one of paragraphs 17 to 26, wherein using the subject's cognitive information to compute the probability is performed after using the subject's clinical information.
28. A method of treating a patient diagnosed with psychosis using the CIM of any one of paragraphs 1 to 16, wherein the treatment includes any one or more of antipsychotic medications (typical and atypical), mood stabilizers, anxiolytics, antidepressants, cognitive behavioral therapy for psychosis, supportive therapy, insight-oriented therapy, family therapy, social skills training, vocational rehabilitation, case management, hospitalization, electroconvulsive therapy (ECT), sleep hygiene, exercise, dietary adjustments, mindfulness, relaxation techniques, or any combinations thereof.
29. The method of paragraph 28, wherein the antipsychotic medication is selected from haloperidol, chlorpromazine, fluphenazine, perphenazine, thioridazine, trifluoperazine, loxapine, pimozide, risperidone, olanzapine, quetiapine, aripiprazole, clozapine, ziprasidone, paliperidone, lurasidone, asenapine, brexpiprazole, cariprazine, or a combination thereof.
30. A method of treating a patient diagnosed with psychosis using a device containing the non-transitory CRM of any one of paragraphs 17 to 27, wherein the treatment includes any one or more of antipsychotic medications (typical and atypical), mood stabilizers, anxiolytics, antidepressants, cognitive behavioral therapy for psychosis, supportive therapy, insight-oriented therapy, family therapy, social skills training, vocational rehabilitation, case management, hospitalization, electroconvulsive therapy (ECT), sleep hygiene, exercise, dietary adjustments, mindfulness, relaxation techniques, or any combinations thereof.
31. The method of paragraph 30, wherein the antipsychotic medication is selected from haloperidol, chlorpromazine, fluphenazine, perphenazine, thioridazine, trifluoperazine, loxapine, pimozide, risperidone, olanzapine, quetiapine, aripiprazole, clozapine, ziprasidone, paliperidone, lurasidone, asenapine, brexpiprazole, cariprazine, or a combination thereof.
32. A computer-aided method for neurobiological diagnosis of a patient with an idiopathic psychosis, with that diagnosis having corresponding treatment targets, with the method involving:
   a. receiving measured clinical, cognitive, and behavioral performance data of the patient;
   b. processing the measured clinical, cognitive, and behavioral performances and signals of the patient;
   c. obtaining clinical features and laboratory outcomes from the measured and processed clinical, behavioral, and cognitive performances and signals;
   d. applying an adaptive diagnostic algorithm to the obtained clinical feature and laboratory outcomes to obtain a B-SNIP psychosis Biotype diagnosis on the patient;
   e. applying a corresponding targeted treatment for the patient based upon the obtained B-SNIP psychosis Biotype; and
   f. applying the processed measured scales, signals, and one or more new biomarkers to one or more new patients to obtain one or more updated bio-factors and re-tune the adaptive diagnosis algorithm.
33. The method of paragraph 32, wherein a related Biotypes algorithm, is trained on a large multivariate biomarker dataset equal to greater than one thousand observations of idiopathic psychosis cases, and wherein, prior to re-tuning the related Biotypes algorithm, continuously adding additional idiopathic psychosis cases and novel measures to the related Biotype algorithm.
34. The method of paragraph 32 or 33, wherein collecting measured characteristics from the patient includes:
   measuring clinical features of the patient;
   measuring saccades and anti-saccades;
   measuring motor inhibition on the patient; and/or
   measuring cognitive performance on the patient.
35. The method of any one of paragraphs 32 to 34, wherein the B-SNIP psychosis Biotypes comprise a neural dysregulation Biotype (BT2), a neural vigor Biotype (BT1), and a stimulus salience Biotype (BT3).
36. The method of any one of paragraphs 32 to 35, wherein the B-SNIP psychosis Biotypes are used to implement treatments specific to corresponding patterns of neurobiological deviations.
37. A system for assigning a neurobiological diagnosis of a patient with an idiopathic psychosis, with that diagnosis having corresponding treatment targets, the system containing:
   a. one or more processors; and
   b. tangible, non-transitory memories configured to communicate with the one or more processors, the tangible, non-transitory memories having instructions stored thereon that, in response to execution by the one or more processors, cause the one or more processors to perform operations involving:
      i. measuring clinical characteristics of the patient;
      ii. measuring behavioral performance of the patient;
      iii. measuring cognitive performance of the patient;
      iv. processing the measured clinical, behavioral, and cognitive performances and signals;
      v. feeding the measured and processed clinical, behavioral, and cognitive performances and signals and an adaptive diagnosis algorithm to obtain a B-SNIP psychosis Biotype;
      vi. applying a corresponding targeted treatment for the patient based upon the obtained psychosis Biotype; and/or
      vii. applying the processed measured characteristics, scales, signals, and one or more new biomarkers to one or more new patients to obtain updated bio-factors and re-tune the adaptive diagnosis algorithm.
38. The system of paragraph 37, wherein a related Biotypes algorithm is trained on a multivariate biomarker dataset of at least one thousand idiopathic psychosis cases, and wherein, prior to re-tuning the related Biotypes algorithm, adding additional idiopathic psychosis cases and novel measures to the related Biotype algorithm.
39. The system of paragraph 37 or 38, wherein the adaptive diagnosis algorithm is re-tuned based on the re-tuned Biotypes algorithm.
40. The system of any one of paragraphs 37 to 39, wherein collecting measured signals of the patient includes:
   measuring clinical characteristics;
   measuring saccades and anti-saccades;
   measuring motor inhibition on the patient; and/or
   measuring cognitive performance on the patient.
41. The system of paragraph 40, wherein those measured signals of the patient are input to the adaptive diagnosis algorithm to obtain a Biotype diagnosis
42. The system of paragraph 40, wherein the B-SNIP psychosis Biotypes comprise a neural dysregulation Biotype (BT2), a neural vigor Biotype (BT1), and a stimulus salience Biotype (BT3).
43. The system of paragraph 42, wherein the B-SNIP psychosis Biotypes are used to implement treatments specific to corresponding patterns of neurobiological deviations.
44. A system for diagnosing an idiopathic psychosis and corresponding treatment targeting for a patient, the system containing:
   a. one or more processors configured to:
      1. process received information, wherein the received information comprises measurements of clinical, behavioral, and cognitive performance of the patient; and
   b. tangible, non-transitory memories configured to communicate with the one or more processors, the tangible, non-transitory memories having instructions stored thereon that, in response to execution by the one or more processors, cause the one or more processors to perform operations involving:
      1. applying, via the adaptive diagnosis algorithm, to the processed measured performances and signals of the patient, a numerical taxonomy to classify the patient as belonging to a B-SNIP psychosis Biotype;
      2. applying a corresponding targeted treatment for the patient based upon the psychosis Biotype; and
      3. re-tuning the adaptive diagnosis algorithm using updated processed measured performances and signals, to improve psychosis Biotype diagnoses.
45. A method for diagnosing serious psychiatric conditions, the method involving:
   measuring cognition on a patient using a device configured to present audio data, visual data, audio-visual data, optical data, electrical data, magnetic data, electromagnetic data, mechanical data or a combination thereof;
   measuring saccades and anti-saccades on the patient;
   measuring motor inhibition on the patient;
   measuring EEG signals on the patient;
   using a computing device to apply pre-processing to the measured signals to prepare them for implementation;
   using the computing device or another computing device to perform principal components analysis of the measured signals and scales to determine the most significant components (e.g., called bio-factors);
   applying numerical taxonomy to classify the patient as belonging to a Biotype based on the bio-factors, wherein using the bio-factors leads to enhanced diagnostic accuracy and reduced computational burden than not user the bio-factors;
   using a diagnostic algorithm to diagnose the patient's serious psychiatric condition based on the classified Biotype in preparation for treatment prescription; and
   adding, optionally continually, new idiopathic psychosis cases and new measures to a B-SNIP database prior to and retraining the diagnostic algorithm on a computer to improve its diagnostic precision and enhance its ease and diversity of implementations for treatment selection.

Those skilled in the art will recognize, or be able to ascertain using no more than routine experimentation, many equivalents to the specific embodiments of the innovative work described herein. Such equivalents are intended to be encompassed by the following claims.

We claim:

1. A computer-implemented method (CIM) for neurobiological diagnosis of a subject, optionally followed by an individual devising a treatment regimen, the method comprising:
using one or more adaptive diagnostic algorithms configured to (i) process the subject's clinical information, cognitive information, and/or behavioral information to obtain a first score and (ii) compute and/or output a probability of the subject having a subtype of psychosis by matching the first score with corresponding scores of other subjects with a known subtype of psychosis, wherein the one or more adaptive diagnostic algorithms have been trained on a second set of clinical information, cognitive information, and/or behavioral information to recognize one or more subtypes of psychosis, optionally wherein training of the one or more adaptive diagnostic algorithms occurs on a computing device,
wherein the one or more adaptive diagnostic algorithms are operably linked to one or more processors capable of executing the one or more adaptive diagnostic algorithms, and optionally wherein the psychosis is idiopathic.

2. The CIM of claim 1, wherein the one or more adaptive diagnostic algorithms are operably linked to (i) a device configured to present audio data, visual data, audio-visual data, optical data, electrical data, magnetic data, electromagnetic data, mechanical data or a combination thereof, related to neurobiological diagnosis.

3. The CIM of claim 1, wherein the one or more adaptive diagnostic algorithms are configured to process the subject's clinical information.

4. The CIM of claim 1, wherein the one or more adaptive diagnostic algorithms are configured to process the subject's clinical information and cognitive information.

5. The CIM of claim 1, wherein the one or more adaptive diagnostic algorithms are configured to process the subject's clinical information, cognitive information, and behavioral information.

6. The CIM of claim 1, wherein the one or more adaptive diagnostic algorithms comprise a randomized ensemble of classifiers.

7. The CIM of claim 1, wherein the one or more adaptive diagnostic algorithms comprise an adaptive decision tree algorithm.

8. The CIM of claim 1, wherein the one or more adaptive diagnostic algorithms comprise an extra-trees classifier.

9. The CIM of claim 1, wherein using the subject's cognitive information to compute the probability is performed after using the subject's clinical information.

10. The CIM of claim 1, wherein the subtype of psychosis comprises a neural dysregulation Biotype (BT2), a neural vigor Biotype (BT1), and/or a stimulus salience Biotype (BT3).

11. The CIM of claim 1, further comprising:
causing a recommendation of one or more treatment regiments based on the subtype of psychosis to be presented at a user interface comprising a display of a computing device, an electro-mechanical acoustic system, or a combination thereof.

12. The CIM of claim 1, wherein the CIM provides or is capable of providing neurobiological diagnosis in real-time.

13. The CIM of claim 1, comprising an individual devising a treatment regimen, wherein the treatment regimen comprises antipsychotic medications (typical and atypical), mood stabilizers, anxiolytics, antidepressants; cognitive behavioral therapy for psychosis; supportive therapy; insight-oriented therapy; family therapy; social skills training; vocational rehabilitation; case management; hospitalization; electroconvulsive therapy (ECT); sleep hygiene; exercise; dietary adjustments; mindfulness; relaxation techniques; or any combinations thereof.

14. A non-transitory computer-readable medium (CRM) with one or more computer-executable instructions stored thereon executed by one or more processors, wherein the one or more computer-executable instructions comprise one or more adaptive diagnostic algorithms configured to (i) process a subject's clinical information, cognitive information, and/or behavioral information to obtain a first score and (ii) compute and/or output a probability of the subject having a subtype of psychosis by matching the first score with corresponding scores of other subjects with a known subtype of psychosis, wherein the one or more adaptive diagnostic algorithms have been trained on a second set of clinical information, cognitive information, and/or behavioral information to recognize one or more subtypes of psychosis, optionally wherein training of the one or more adaptive diagnostic algorithms occurs on a computing device, and
wherein the one or more computer-executable instructions are operably linked to the one or more processors.

15. The non-transitory CRM of claim 14, wherein the one or more computer-executable instructions are operably linked to a device configured to receive or output audio data, visual data, audio-visual data, optical data, electrical data, magnetic data, electromagnetic data, mechanical data, or a combination thereof, related to neurobiological diagnosis.

16. The non-transitory CRM of claim 14, wherein the one or more adaptive diagnostic algorithms are configured to process:
(i) the subject's clinical information,
(ii) the subject's clinical information and cognitive information, or
(iii) the subject's clinical information and cognitive information.

17. The non-transitory CRM of claim 14, wherein the one or more adaptive diagnostic algorithms comprise a randomized ensemble of classifiers.

18. The non-transitory CRM claim 14, wherein the one or more adaptive diagnostic algorithms comprise an adaptive decision tree algorithm.

19. A method of treating a patient diagnosed with psychosis using the CIM of claim 1, wherein the treatment comprises any one or more of antipsychotic medications (typical and atypical), mood stabilizers, anxiolytics, antidepressants, cognitive behavioral therapy for psychosis, supportive therapy, insight-oriented therapy, family therapy, social skills training, vocational rehabilitation, case management, hospitalization, electroconvulsive therapy (ECT), sleep hygiene, exercise, dietary adjustments, mindfulness, relaxation techniques, or any combinations thereof.

20. A method of treating a patient diagnosed with psychosis using a device comprising the non-transitory CRM of claim 14, wherein the treatment comprises any one or more of antipsychotic medications (typical and atypical), mood stabilizers, anxiolytics, antidepressants, cognitive behavioral therapy for psychosis, supportive therapy, insight-oriented therapy, family therapy, social skills training, vocational rehabilitation, case management, hospitalization, electroconvulsive therapy (ECT), sleep hygiene, exercise, dietary adjustments, mindfulness, relaxation techniques, or any combinations thereof.
